SEQUENCE LISTING (32 sequences)

SEQ ID NO: 1 - DNA, 16 bp, synthetic construct
  source 1..16: mol_type = other DNA
cttgtaatggtatcac

SEQ ID NO: 2 - DNA, 16 bp, synthetic construct
  source 1..16: mol_type = other DNA
aatggtaccactgggc

SEQ ID NO: 3 - DNA, 15 bp, synthetic construct
  source 1..15: mol_type = other DNA
tgcagaatcaaacca

SEQ ID NO: 4 - DNA, 16 bp, synthetic construct
  source 1..16: mol_type = other DNA
gcctaggtttacaaac

SEQ ID NO: 5 - DNA, 16 bp, synthetic construct
  source 1..16: mol_type = other DNA
gcgtagagctgaaaca

SEQ ID NO: 6 - DNA, 16 bp, synthetic construct
  source 1..16: mol_type = other DNA
gtgcgtagagctgaaa

SEQ ID NO: 7 - DNA, 16 bp, synthetic construct
  source 1..16: mol_type = other DNA
aagtgcgtagagctga

SEQ ID NO: 8 - DNA, 16 bp, synthetic construct
  source 1..16: mol_type = other DNA
gaaagtgcgtagagct

SEQ ID NO: 9 - DNA, 16 bp, synthetic construct
  source 1..16: mol_type = other DNA
ctgaaagtgcgtagag

SEQ ID NO: 10 - DNA, 16 bp, synthetic construct
  source 1..16: mol_type = other DNA
ccctgaaagtgcgtag

SEQ ID NO: 11 - DNA, 16 bp, synthetic construct
  source 1..16: mol_type = other DNA
accctgaaagtgcgta

SEQ ID NO: 12 - DNA, 16 bp, synthetic construct
  source 1..16: mol_type = other DNA
agtaccctgaaagtgc

SEQ ID NO: 13 - DNA, 16 bp, synthetic construct
  source 1..16: mol_type = other DNA
ccctcagtaccctgaa

SEQ ID NO: 14 - DNA, 16 bp, synthetic construct
  source 1..16: mol_type = other DNA
gggattaccgaaatag

SEQ ID NO: 15 - DNA, 15 bp, synthetic construct
  source 1..15: mol_type = other DNA
ctgggattaccgaaa

SEQ ID NO: 16 - DNA, 16 bp, synthetic construct
  source 1..16: mol_type = other DNA
acccaacacctgacac

SEQ ID NO: 17 - DNA, 16 bp, synthetic construct
  source 1..16: mol_type = other DNA
ctaacccaacacctga

SEQ ID NO: 18 - DNA, 16 bp, synthetic construct
  source 1..16: mol_type = other DNA
gccctaacccaacacc

SEQ ID NO: 19 - DNA, 16 bp, synthetic construct
  source 1..16: mol_type = other DNA
atgttctcagcgctga

SEQ ID NO: 20 - DNA, 16 bp, synthetic construct
  source 1..16: mol_type = other DNA
ggagaaccctgaatgt

SEQ ID NO: 21 - DNA, 16 bp, synthetic construct
  source 1..16: mol_type = other DNA
ctgcagaatcaaacca

SEQ ID NO: 22 - DNA, 16 bp, synthetic construct
  source 1..16: mol_type = other DNA
atgttctcagcgctga

SEQ ID NO: 23 - DNA, 16 bp, synthetic construct
  source 1..16: mol_type = other DNA
ctgggattaccgaaat

SEQ ID NO: 24 - DNA, 92 bp, Homo sapiens
  source 1..92: mol_type = genomic DNA
gtatataacagagtttgtaaacctaggcaatgtttcagctctacgcactttcagggtactgagggctttgaaaactatttcggtaatcccag

SEQ ID NO: 25 - DNA, 92 bp, Homo sapiens
  source 1..92: mol_type = genomic DNA
atatgtgacagagtttgtggacctgggcaatgtctcagcgctgagaacattcagggttctccgagctttgaaaactatctctgtaattccag

SEQ ID NO: 26 - DNA, 23 bp, synthetic construct
  source 1..23: mol_type = other DNA
tggaactggttagatttcagtgt

SEQ ID NO: 27 - DNA, 22 bp, synthetic construct
  source 1..22: mol_type = other DNA
caacacacttgtttcgaaggtt

SEQ ID NO: 28 - RNA, 16 nt, synthetic construct
  source 1..16: mol_type = other RNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified thymine with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified thymine with phosphorothioate linkage
  modified_base 5: mod_base = OTHER; note = 2'-O-methoxyethyl modified thymine with phosphorothioate linkage
  modified_base 6: mod_base = OTHER; note = 2'-O-methoxyethyl modified nucleotide with phosphorothioate linkage
  modified_base 7: mod_base = OTHER; note = 2'-O-methoxyethyl modified thymine with phosphorothioate linkage
  modified_base 8: mod_base = OTHER; note = 2'-O-methoxyethyl modified nucleotide with phosphorothioate linkage
  modified_base 9: mod_base = OTHER; note = 2'-O-methoxyethyl modified nucleotide with phosphorothioate linkage
  modified_base 10: mod_base = OTHER; note = 2'-O-methoxyethyl modified nucleotide with phosphorothioate linkage
  modified_base 11: mod_base = OTHER; note = 2'-O-methoxyethyl modified nucleotide with phosphorothioate linkage
  modified_base 12: mod_base = OTHER; note = 2'-O-methoxyethyl modified nucleotide with phosphorothioate linkage
  modified_base 13: mod_base = OTHER; note = 2'-O-methoxyethyl modified nucleotide with phosphorothioate linkage
  modified_base 14: mod_base = OTHER; note = 2'-O-methoxyethyl modified thymine with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl modified nucleotide
atgttctcagcgctga

SEQ ID NO: 29 - RNA, 16 nt, synthetic construct
  source 1..16: mol_type = other RNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = 2'-O-methoxyethyl modified nucleotide with phosphorothioate linkage
  modified_base 7: mod_base = OTHER; note = 2'-O-methoxyethyl modified nucleotide with phosphorothioate linkage
  modified_base 8: mod_base = OTHER; note = 2'-O-methoxyethyl modified nucleotide with phosphorothioate linkage
  modified_base 9: mod_base = OTHER; note = 2'-O-methoxyethyl modified nucleotide with phosphorothioate linkage
  modified_base 10: mod_base = OTHER; note = 2'-O-methoxyethyl modified thymine with phosphorothioate linkage
  modified_base 11: mod_base = OTHER; note = 2'-O-methoxyethyl modified nucleotide with phosphorothioate linkage
  modified_base 12: mod_base = OTHER; note = 2'-O-methoxyethyl modified nucleotide with phosphorothioate linkage
  modified_base 13: mod_base = OTHER; note = 2'-O-methoxyethyl modified nucleotide with phosphorothioate linkage
  modified_base 14: mod_base = OTHER; note = 2'-O-methoxyethyl modified thymine with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl modified thymine
ggagaaccctgaatgt

SEQ ID NO: 30 - DNA, 92 bp, Homo sapiens
  source 1..92: mol_type = genomic DNA
atatataacagagtttgtaaacctaggcaatgtttcagctctacgcactttcagggtactgagggctttgaaaactatttcggtaatcccag

SEQ ID NO: 31 - protein, 31 aa, Homo sapiens
YVTEFVDLGNVSALRTFRVLRALKTISVIPG

SEQ ID NO: 32 - protein, 31 aa, Homo sapiens
YITEFVNLGNVSALRTFRVLRALKTISVIPG

(19) United States
(12) Patent Application Publication
(10) Pub. No.: US 2026/0159844 A1
(43) Pub. Date: Jun. 11, 2026

(54) METHODS AND COMPOSITIONS FOR TREATING NEUROLOGICAL DISEASES AND DISORDERS AND CANCERS

(71) Applicant: Trustees of Tufts College, Medford, MA (US)

(72) Inventors: Madeleine Oudin, Medford, MA (US); Haley Dame, Medford, MA (US)

(73) Assignee: Trustees of Tufts College, Medford, MA (US)

(21) Appl. No.: 19/409,204

(22) Filed: Dec. 4, 2025

Related U.S. Application Data
(63) Continuation of application No. PCT/US2024/032449, filed on Jun. 4, 2024.
(60) Provisional application No. 63/471,030, filed on Jun. 5, 2023.

(51) Int. Cl. C12N 15/113; A61K 31/712; A61K 31/7125

(52) U.S. Cl. CPC C12N 15/1138; A61K 31/712; A61K 31/7125; C12N 2310/11; C12N 2310/315; C12N 2310/322; C12N 2320/33

(57) ABSTRACT

Provided herein are antisense oligonucleotides and therapeutic methods featuring such oligonucleotides. These oligonucleotides and methods are useful for promoting switching of exon 5 isoforms of a gene or polynucleotide encoding a voltage gated sodium ion channel polypeptide and for treating a neurological, neurodevelopmental, or neurodegenerative disorder, disease, or pathology, or a cancer, and/or symptoms thereof in a subject.

### CROSS REFERENCE TO RELATED APPLICATIONS

This application is a continuation under 35 U.S.C. § 111(a) of PCT International Patent Application No. PCT/US2024/032449, filed Jun. 4, 2024, designating the United States and published in English, which claims priority to and the benefit of U.S. Provisional Application No. 63/471,030, filed Jun. 5, 2023, the entire contents of each of which are incorporated by reference herein.

### STATEMENT OF FEDERALLY FUNDED RESEARCH

This invention was made with government support under Grant No. CA271387 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Jun. 24, 2024, is named 167774-013101_PCT_SL.xml and is 48,320 bytes in size.

### BACKGROUND

Genetic variation in the genes encoding voltage-gated sodium ion channels are associated with, and can be causative of, neurological diseases and disorders, as well as cancer.

Sodium ion channel genes are composed of multiple exons, and differing combinations of these exons result in gene isoforms, which can change the amino acid sequence of the encoded proteins (protein isoforms or proteoforms). Even relatively small changes in protein sequence and structure of the sodium channel isoforms can have significant effects on the function of the channel. Splice isoforms of the sodium channels can alter the electrophysiological characteristics and biophysical properties of the encoded channels, as well as the functional impact of the isoform variants associated with disease and dysfunction.

Across the developmental stages of an individual, the functional role, subcellular location, expression level, and exon isoform selection of voltage-gated sodium channels vary. Channelopathies are diseases that develop because of defects or dysfunction of cell membrane ion channels and have been implicated in a wide variety of diseases consistent with the distribution of ion channels throughout the body.

A need exists to understand how isoform differences modify the function of an encoded voltage-gated sodium ion channel, as well as the etiology of the diseases and disorders associated with channel isoforms, the dysfunction of sodium channels (channelopathies), and their therapeutic management. Agents and methods for treating diseases, disorders and pathologies associated with sodium ion channel mutations and channelopathies are urgently required.

### SUMMARY

Described and featured herein are antisense oligonucleotides (ASOs) that regulate the splicing and promote alternative splicing of exon 5 of a voltage-gate sodium ion channel protein-encoding gene. In an embodiment, the voltage-gate sodium ion channel protein-encoding gene is SCN8A, which encodes the Nav1.6 sodium channel. In an embodiment, the voltage-gate sodium ion channel protein-encoding gene is SCN5A, which encodes the Nav1.5 sodium channel. Exon 5 of the SCN8A gene, as well as other sodium ion channel-encoding genes described herein, constitutes two isoforms: a 5N exon isoform, which has been associated with neurological, neurodevelopmental and neurodegenerative disorders and diseases, such as, for example, epilepsy, epileptic seizures, epileptic encephalopathy, early infantile epileptic encephalopathy, etc., as well as cancer progression, and a 5A exon isoform, which has been associated with various cancers, for example, breast cancer and metastatic breast cancer, as well as with certain neurological, neurodevelopmental and neurodegenerative disorders and diseases, such as, for example, epilepsy. Increased sodium ion channel (e.g., Nav1.6) activity has been linked to exon 5N. Mutations or alterations in exon 5A and in exon 5N have been associated with epilepsy. The ASOs as described herein may be used as therapeutics for the treatment of neuronal, neurological, neurodevelopmental and neurodegenerative disorders and diseases, and/or cancers.

In an aspect, an antisense oligonucleotide comprising 8-20 nucleobases, wherein at least 90% of the nucleobases or more than 8 consecutive nucleobases of the oligonucleotide are complementary to a nucleic acid sequence in an isoform of exon 5 of an SCN8A gene encoding a voltage-gated sodium ion channel polypeptide is provided. In an embodiment, the isoform of exon 5 is the exon 5N isoform. In an embodiment, the isoform of exon 5 is the exon 5A isoform. In an embodiment, the antisense oligonucleotide comprises 10-16 consecutive nucleobases. In an embodiment, the voltage-gated sodium ion channel encoded by the SCN8A gene is Nav1.6. In an embodiment, the antisense oligonucleotide comprises or consists of a nucleic acid sequence having at least 80% (80% or greater) sequence identity to an ASO nucleotide sequence selected from the group listed in Table 1 or Table 2. In an embodiment, the antisense oligonucleotide comprises or consists of a nucleic acid sequence having at least 90% (90% or greater) sequence identity to an ASO nucleotide sequence selected from the group listed in Table 1 or Table 2. In an embodiment, the antisense oligonucleotide (ASO) comprises or consists of a nucleic acid sequence having complete sequence identity to a nucleotide sequence selected from the ASOs listed in Table 1 or Table 2. In an embodiment, the antisense oligonucleotide (ASO) comprises at least 10 consecutive nucleobases of the following nucleic acid sequences, or comprises or consists of the following nucleic acid sequences: cttgtaatggtatcac; aatggtaccactgggc; tgcagaatcaaacca; gcctaggtttacaaac; gcgtagagctgaaaca; gtgcgtagagctgaaa; aagtgcgtagagctga; gaaagtgcgtagagct; ctgaaagtgcgtagag; ccctgaaagtgcgtag; accctgaaagtgcgta; agtaccctgaaagtgc; ccctcagtaccctgaa; gggattaccgaaatag; ctgggattaccgaaa; acccaacacctgacac; ctaacccaacacctga; gccctaacccaacacc, atgttctcagcgctga; or ggagaaccctgaatgt. In an embodiment, the antisense oligonucleotide (ASO) comprises or consists of a nucleic acid sequence selected from ctgcagaatcaaacca; gcgtagagctgaaaca; gtgcgtagagctgaaa; aagtgcgtagagctga; gaaagtgcgtagagct; ctgaaagtgcgtagag; ccctgaaagtgcgtag; accctgaaagtgcgta; agtaccctgaaagtgc; or ccctcagtaccctgaa. In an embodiment, the antisense oligonucleotide (ASO) comprises or consists of a nucleic acid sequence selected from ctgcagaatcaaacca; gaaagtgcgtagagct; ctgaaagtgcgtagag; ccctgaaagtgcgtag; or agtaccctgaaagtgc. In an embodiment, the antisense oligonucleotide (ASO) comprises or consists of nucleic acid sequence atgttctcagcgctga or nucleic acid sequence ggagaaccctgaatgt.

Another aspect provides an isolated or purified antisense oligonucleotide (ASO) for modifying pre-mRNA splicing of exon 5 in the SCN8A sodium ion channel-encoding gene which specifically modulates splicing of the SCN8A exon 5N transcript. In an embodiment, the ASO is ASO-3, which comprises or consists of nucleic acid sequence ctgcagaatcaaacca; ASO-8, which comprises or consists of nucleic acid sequence gaaagtgcgtagagct; ASO-9, which comprises or consists of nucleic acid sequence ctgaaagtgcgtagag; ASO-10, which comprises or consists of nucleic acid sequence ccctgaaagtgcgtag; ASO-12, which comprises or consists of nucleic acid sequence agtaccctgaaagtgc, or ASO-13, which comprises or consists of nucleic acid sequence ccctcagtaccctgaa. In an embodiment, the modulation of pre-mRNA splicing induces exclusion of exon 5N of SCN8A RNA.

Another aspect provides an isolated or purified antisense oligonucleotide (ASO) for modifying pre-mRNA splicing of exon 5 in the SCN8A sodium ion channel-encoding gene which specifically modulates splicing of the SCN8A exon 5A transcript. In an embodiment, the ASO is ASO-24, which comprises or consists of nucleic acid sequence atgttctcagcgctga. In an embodiment, the ASO is ASO-27, which comprises or consists of nucleic acid sequence ggagaaccctgaatgt. In an embodiment, the modulation of pre-mRNA splicing induces exclusion of exon 5A of SCN8A RNA.

In an embodiment of any one of the above-delineated aspects and/or embodiments thereof, the antisense oligonucleotide comprises a modified linkage selected from the group consisting of phosphorothioate, methylphosphonate, phosphodiester, phosphotriester, and phosphorodithioate linkages. In an embodiment, the modified linkage is a phosphorothioate linkage. In an embodiment or any one of the above-delineated aspects and/or embodiments thereof, the antisense oligonucleotide further includes at least one modified sugar moiety. In an embodiment, the modified sugar moiety is a 2′-O-methoxyethyl (2′-MOE) group, a 2′-O-methyl, a 2′-dimethylaminooxyethoxy, a 2′-dimethylaminoethoxyethoxy, a 2′-fluoro, or a 2′-acetamide modification group. In an embodiment, the modified sugar moiety is a 2′-O-methoxyethyl (2′-MOE) group.

In another aspect, an isolated or purified antisense oligonucleotide (ASO) for modifying pre-mRNA splicing of exon 5 in the SCN5A sodium ion channel-encoding gene which specifically modulates splicing of the SCN5A exon 5N transcript is provided. In an embodiment, the ASO is ASO-10, which comprises or consists of nucleic acid sequence ccctgaaagtgcgtag; ASO-11, which comprises or consists of nucleic acid sequence accctgaaagtgcgta; or ASO-13, which comprises or consists of nucleic acid sequence ccctcagtaccctgaa. In an embodiment, the ASO specifically targets, binds, and modulates alternative splicing of SCN5A RNA, thereby specifically decreasing the inclusion of exon 5N of the SCN5A gene. In an embodiment, the oligonucleotide comprises a modified linkage which is a phosphorothioate linkage. In an embodiment, the oligonucleotide comprises a modified sugar moiety which is a 2′-O-methoxyethyl (2′-MOE) group.

In another aspect, a set or panel of antisense oligonucleotides comprising two or more of the antisense oligonucleotides of any of the above-delineated aspects and/or embodiments thereof is provided. In embodiments, the set or panel comprises two or more of the ASOs as set forth in Table 1 and/or Table 2.

In another aspect, a pharmaceutical composition comprising an effective amount of the antisense oligonucleotide of any of the above-delineated aspects and/or embodiments thereof and a pharmaceutically acceptable excipient, diluent, or carrier is provided. In an embodiment, the pharmaceutical composition comprises one or more of the ASOs as set forth in Table 1 and/or Table 2.

In another aspect, a method of downregulating the activity of an exon 5 isoform of an SCN8A sodium ion channel gene in a cell is provided, in which the method involves contacting the cell with an effective amount of an isolated or purified antisense oligonucleotide (ASO) of any of the above-delineated aspects and embodiments thereof, thereby downregulating the exon 5 isoform activity. In embodiments, the practice of the method modifies pre-mRNA splicing of exon 5 in the SCN8A sodium ion channel-encoding gene which specifically modulates or affects splicing of the SCN8A exon 5N or the SCN8A exon 5A transcript. In an embodiment, the ASO is selected from ASO-3, which comprises or consists of nucleic acid sequence ctgcagaatcaaacca; ASO-8, which comprises or consists of nucleic acid sequence gaaagtgcgtagagct; ASO-9, which comprises or consists of nucleic acid sequence ctgaaagtgcgtagag; ASO-10, which comprises or consists of nucleic acid sequence ccctgaaagtgcgtag; ASO-12, which comprises or consists of nucleic acid sequence agtaccctgaaagtgc; or ASO-13, which comprises of consists of nucleic acid sequence ccctcagtaccctgaa. In an embodiment, the ASO modulates pre-mRNA splicing to induce the exclusion of exon 5N of SCN8A RNA. In an embodiment, the ASO modulates pre-mRNA splicing to induce the exclusion of exon 5A of SCN8A RNA. In an embodiment, the ASO is ASO-24, which comprises or consists of nucleic acid sequence atgttctcagcgctga. In an embodiment, the ASO is ASO-27, which comprises or consists of nucleic acid sequence ggagaaccctgaatgt. In an embodiment, the ASO comprises a modified linkage selected from the group consisting of phosphorothioate, methylphosphonate, phosphodiester, phosphotriester, and phosphorodithioate linkages. In an embodiment, the modified linkage is a phosphorothioate linkage. In an embodiment, the ASO further comprises at least one modified sugar moiety. In embodiments, the modified sugar moiety is a 2′-O-methoxyethyl (2′-MOE) group, a 2′-O-methyl, a 2′-dimethylaminooxyethoxy, a 2′-dimethylaminoethoxyethoxy, a 2′-fluoro, or a 2′-acetamide modification group. In an embodiment, the modified sugar moiety is a 2′-O-methoxyethyl (2′-MOE) group. In another embodiment of the method, the exon 5 isoform is an exon 5N isoform or an exon 5A isoform. In an embodiment of the method, the antisense oligonucleotide induces a decrease in exon 5N isoform expression of the SCN8A gene. In an embodiment of the method, the antisense oligonucleotide induces a decrease in exon 5A isoform expression of the SCN8A gene. In an embodiment of the method, the activity of an Nav1.6 sodium ion channel encoded by SCN8A exon 5N isoform or SCN8A exon 5A isoform is downregulated. In an embodiment of the method, the SCN8A gene contains a mutation or genetic alteration, which can include a missense variant or a gain-of-function mutation or alteration. In an embodiment of the method, the cell is contacted with an effective amount of the antisense oligonucleotide of any one of the above-delineated aspects and/or embodiments thereof.

In another aspect, a method of altering RNA splicing of exon 5 of a gene encoding a voltage gated sodium ion channel protein in a cell is provided, in which the method involves introducing into the cell an effective amount of the antisense oligonucleotide of any one of the above-delineated aspects and/or embodiments thereof, thereby altering RNA splicing by inducing a switch from exon 5N to exon 5A expression and transcription or altering RNA splicing by inducing a switch from exon 5A to exon 5N expression and transcription. In an embodiment, the gene is SCN8A. In an embodiment, the SCN8A gene comprises a missense variant or a gain-of-function mutation or alteration. An embodiment of the method provides an effective amount of the isolated or purified antisense oligonucleotide (ASO) for modifying pre-mRNA splicing of exon 5 in the SCN8A sodium ion channel-encoding gene which specifically modulates splicing of the SCN8A exon 5N transcript or the ASO selected from ASO-3, which comprises or consists of nucleic acid sequence ctgcagaatcaaacca; ASO-8, which comprises or consists of nucleic acid sequence gaaagtgcgtagagct; ASO-9, which comprises or consists of nucleic acid sequence ctgaaagtgcgtagag; ASO-10, which comprises or consists of nucleic acid sequence ccctgaaagtgcgtag; or ASO-12, which comprises or consists of nucleic acid sequence agtaccctgaaagtgc, or a pharmaceutically acceptable composition thereof, introduced into the cell. Another embodiment of the method provides an effective amount of the isolated or purified antisense oligonucleotide (ASO) for modifying pre-mRNA splicing of exon 5 in the SCN8A sodium ion channel-encoding gene which specifically modulates splicing of the SCN8A exon 5A transcript or the ASO selected from ASO-24, which comprises or consists of nucleic acid sequence atgttctcagcgctga, or ASO-27, which comprises or consists of nucleic acid sequence ggagaaccctgaatgt, introduced into the cell. In an embodiment, the method increases inclusion of exon 5A or increases the inclusion of exon 5N in Nav1.6 in the cell, thereby reducing overall activity of Nav1.6 in the cell. In embodiments, the cell is a neuronal cell, a neuron, a primary cortical neuron, a brain cell, or a cell of the central nervous system. In embodiments, the cell is in vivo, in vitro, or ex vivo.

In another aspect, a method of treating a neuronal, neurological, neurodevelopmental, or neurodegenerative disorder, disease, or pathology or symptoms thereof, associated with a variant or mutated gene encoding a voltage gated sodium ion channel protein in a subject is provided, in which the method involves administering to the subject an effective amount of the antisense oligonucleotide of any one of the above-delineated aspects and/or embodiments thereof, or a pharmaceutically acceptable composition thereof. In an embodiment, the gene is SCN8A. In an embodiment, the gene is an SCN8A missense variant or contains a gain-of-function mutation or alteration. An embodiment of the method provides an effective amount of the isolated or purified antisense oligonucleotide (ASO) for modifying pre-mRNA splicing of exon 5 in the SCN8A sodium ion channel-encoding gene which specifically modulates splicing of the SCN8A exon 5N transcript or the ASO selected from ASO-3, which comprises or consists of nucleic acid sequence ctgcagaatcaaacca; ASO-8, which comprises or consists of nucleic acid sequence gaaagtgcgtagagct; ASO-9, which comprises or consists of nucleic acid sequence ctgaaagtgcgtagag; ASO-10, which comprises or consists of nucleic acid sequence ccctgaaagtgcgtag; ASO-11, which comprises or consists of nucleic acid sequence accctgaaagtgcgta; ASO-12, which comprises or consists of nucleic acid sequence agtaccctgaaagtgc, ASO-13, which comprises or consists of nucleic acid sequence ccctcagtaccctgaa, or a pharmaceutically acceptable composition thereof, administered to the subject. An embodiment of the method provides an effective amount of the isolated or purified antisense oligonucleotide (ASO) for modifying pre-mRNA splicing of exon 5 in the SCN8A sodium ion channel-encoding gene which specifically modulates splicing of the SCN8A exon 5A transcript or the ASO selected from ASO-24, which comprises or consists of nucleic acid sequence atgttctcagcgctga; or ASO-27, which comprises or consists of nucleic acid sequence ggagaaccctgaatgt, or a pharmaceutically acceptable composition thereof, administered to the subject. In embodiments of the method, the neuronal, neurological, neurodevelopmental, or neurodegenerative disorder, disease, or pathology or symptoms thereof is one or more of epilepsy, epileptic seizures, epileptic encephalopathy, early infantile epileptic encephalopathy, developmental delay, autism, autism spectrum disorder (ASDM mild to severe intellectual disabilities, autism spectrum disorders, movement disorders, developmental and epileptic encephalopathy (DEE), severe myoclonic epilepsy of infancy (SMEI)-borderland (SMEB; also known as borderline SMEI); febrile seizure (FS); epilepsy, generalized, with febrile seizures plus (GEFS+); cryptogenic generalized epilepsy; cryptogenic focal epilepsy; myoclonic-astatic epilepsy; Lennox-Gastaut syndrome; idiopathic spasms; sudden unexpected death in epilepsy (SUDEP); or malignant migrating partial seizures of infancy. In an embodiment, the subject is a mammal. In an embodiment, the mammalian subject is a human patient.

In another aspect, a method of inducing an alteration in RNA splicing of exon 5 isoforms of a gene encoding a voltage-gated sodium ion channel polypeptide in a cell of a subject is provided, in which the method involves administering to the subject an effective amount of the antisense oligonucleotide of any one of the above-delineated aspects and/or embodiments thereof, or a pharmaceutically acceptable composition thereof; and wherein the antisense oligonucleotide promotes alternative splicing of an exon 5N isoform to an exon 5A isoform in the cell of the subject, thereby decreasing inclusion of the exon 5N isoform and increasing inclusion of the exon 5A isoform in the voltage-gated sodium ion channel-encoding gene. Alternatively, in the method, the antisense oligonucleotide, or a pharmaceutically acceptable composition thereof, promotes alternative splicing of an exon 5A isoform to an exon 5N isoform in the cell of the subject, thereby decreasing inclusion of the exon 5A isoform and increasing inclusion of the exon 5N isoform in the voltage-gated sodium ion channel-encoding gene. In an embodiment of the method, the gene encoding a voltage-gated sodium ion channel polypeptide is SCN8A and the voltage-gated sodium ion channel polypeptide is Nav1.6. In embodiments of the method, the voltage-gated sodium ion channel-encoding gene is a missense variant or contains a gain-of-function mutation or alteration. An embodiment of the method provides an effective amount of the isolated or purified antisense oligonucleotide (ASO) for modifying pre-mRNA splicing of exon 5 in the SCN8A sodium ion channel-encoding gene which specifically modulates splicing of the SCN8A exon 5A transcript or the ASO selected from ASO-24, which comprises or consists of nucleic acid sequence atgttctcagcgctga of ASO-27, which comprises or consists of nucleic acid sequence ggagaaccctgaatgt, or a pharmaceutically acceptable composition thereof, administered to the subject. In embodiments of the method, the subject has a neuronal, neurological, neurodevelopmental, or neurodegenerative disorder, disease, or pathology or symptoms thereof selected from one or more of epilepsy, epileptic seizures, epileptic encephalopathy, early infantile epileptic encephalopathy, developmental delay, autism, autism spectrum disorder (ASD), mild to severe intellectual disabilities, autism spectrum disorders, movement disorders, developmental and epileptic encephalopathy (DEE), severe myoclonic epilepsy of infancy (SMEI)-borderland (SMEB; also known as borderline SMEI); febrile seizure (FS); epilepsy, generalized, with febrile seizures plus (GEFS+); cryptogenic generalized epilepsy; cryptogenic focal epilepsy; myoclonic-astatic epilepsy; Lennox-Gastaut syndrome; idiopathic spasms; sudden unexpected death in epilepsy (SUDEP); or malignant migrating partial seizures of infancy. In an embodiment of the method, the subject is a mammal. In an embodiment, the mammalian subject is a human patient. In embodiments of the method, the antisense oligonucleotide or the pharmaceutical composition is administered by intrathecal injection or intracerebroventricular injection.

In another aspect, a method of treating a disease or condition in a subject in need thereof by altering RNA splicing of exon 5 of an SCN8A polynucleotide in a cell of the subject is provided, in which the method involves introducing into the cell of the subject an isolated or purified antisense oligonucleotide (ASO), or a pharmaceutically acceptable composition thereof, wherein the antisense oligonucleotide alters RNA splicing of exon 5 of an SCN8A polynucleotide in the cell of the subject and promotes a decrease in inclusion of exon 5N of the SCN8A polynucleotide in the cell or promotes a decrease in inclusion of exon 5A of the SCN8A polynucleotide in the cell. In an embodiment, the ASO modifies or alters pre-mRNA splicing of exon 5 in the SCN8A sodium ion channel-encoding gene which specifically modulates splicing of the SCN8A exon 5N transcript or of the SCN8A exon 5A transcript. In an embodiment, the ASO is selected from ASO-3, which comprises or consists of nucleic acid sequence ctgcagaatcaaacca; ASO-8, which comprises or consists of nucleic acid sequence gaaagtgcgtagagct; ASO-9, which comprises or consists of nucleic acid sequence ctgaaagtgcgtagag; ASO-10, which comprises or consists of nucleic acid sequence ccctgaaagtgcgtag; or ASO-12, which comprises or consists of nucleic acid sequence agtaccctgaaagtgc. In an embodiment, the ASO is ASO-11, which comprises or consists of nucleic acid sequence accctgaaagtgcgta or ASO-13, which comprises or consists of nucleic acid sequence ccctcagtaccctgaa. In an embodiment, the ASO modulates pre-mRNA splicing to induce the exclusion of exon 5N or of exon 5A of SCN8A RNA. In an embodiment of the method, exon 5N inclusion decreases from 83% to an average of 16% in the cell. In embodiments of the method, the cell is a neuronal cell, a neuron, a primary cortical neuron, a brain cell, or a cell of the central nervous system. In embodiments of the method, the disease or condition is selected from one or more of epilepsy, epileptic seizures, epileptic encephalopathy, early infantile epileptic encephalopathy, developmental delay, autism, autism spectrum disorder (ASD), mild to severe intellectual disabilities, autism spectrum disorders, movement disorders, developmental and epileptic encephalopathy (DEE), severe myoclonic epilepsy of infancy (SMEI)-borderland (SMEB; also known as borderline SMEI); febrile seizure (FS); epilepsy, generalized, with febrile seizures plus (GEFS+); cryptogenic generalized epilepsy; cryptogenic focal epilepsy; myoclonic-astatic epilepsy; Lennox-Gastaut syndrome; idiopathic spasms; sudden unexpected death in epilepsy (SUDEP); or malignant migrating partial seizures of infancy.

In another aspect, a method of treating a disease or condition in a subject in need thereof by altering RNA splicing of exon 5 of an SCN5A polynucleotide in a cell of the subject is provided, in which the method includes introducing into the cell of the subject an isolated or purified antisense oligonucleotide (ASO), or a pharmaceutically acceptable composition thereof, wherein the antisense oligonucleotide alters RNA splicing of exon 5 of an SCN5A polynucleotide in the cell of the subject and promotes a decrease in inclusion of exon 5N of the SCN5A polynucleotide in the cell. In an embodiment, the ASO modifies or alters pre-mRNA splicing of exon 5 in the SCN5A sodium ion channel-encoding gene which specifically modulates or alters splicing of the SCN5A exon 5N transcript. In an embodiment, the ASO is ASO-10, which comprises or consists of nucleic acid sequence ccctgaaagtgcgtag; ASO-11, which comprises or consists of nucleic acid sequence accctgaaagtgcgta; or ASO-13, which comprises or consists of nucleic acid sequence ccctcagtaccctgaa. In an embodiment, the ASO specifically targets, binds, and modulates alternative splicing of SCN5A RNA, thereby specifically decreasing the inclusion of exon 5N of the SCN5A gene. In embodiments, the cell is in vivo, in vitro, or ex vivo. In embodiments, the subject is a mammal or a human patient. In an embodiment, an effective amount of the antisense oligonucleotide or a pharmaceutically acceptable composition thereof, is introduced into the cell.

In another aspect, a method of promoting transcription of exon 5A or exon 5N of a gene encoding a voltage gated sodium ion channel protein in a subject in need thereof is provided, in which the method involves administering a therapeutic agent to the subject, wherein the therapeutic agent promotes exclusion of exon 5N or exon 5A transcription in the cell and promotes transcription of exon 5A or exon 5N and expression of the voltage gated sodium ion channel protein encoded by exon 5A or exon 5N; and wherein the therapeutic agent is an antisense oligonucleotide of any one of the above-delineated aspects and/or embodiments thereof, or a pharmaceutically acceptable composition thereof. In an embodiment of the method, the therapeutic agent is an isolated or purified antisense oligonucleotide (ASO) for modifying pre-mRNA splicing of exon 5 in the SCN8A sodium ion channel-encoding gene which specifically modulates splicing of the SCN8A exon 5N or an exon 5A transcript, or a pharmaceutically acceptable composition thereof. In an embodiment, the ASO comprises or consists of a sequence selected from ASO-3 of sequence ctgcagaatcaaacca; ASO-8 of sequence gaaagtgcgtagagct; ASO-9 of sequence ctgaaagtgcgtagag; ASO-10 of sequence ccctgaaagtgcgtag; or ASO-12 of sequence agtaccctgaaagtgc. In an embodiment, the ASO modulates pre-mRNA splicing to induce the exclusion of exon 5N of SCN8A RNA or to induce the exclusion of exon 5A of SCN8A RNA. In an embodiment of the method, the gene encoding a voltage gated sodium ion channel protein is SCN8A and the voltage gated sodium ion channel protein is Nav1.6. In an embodiment, the gene contains a mutation or genetic alteration. In an embodiment, the mutation or alteration is a gain-of-function mutation or alteration. In an embodiment, the gene encoding a voltage gated sodium ion channel protein is SCN5A and the voltage gated sodium ion channel protein is Nav1.5. In an embodiment of the method, the therapeutic agent is introduced into a cell of the subject. In embodiments, the cell is a neuronal cell, a neuron, a brain cell, or a cell of the central nervous system. In embodiments of the method, the antisense oligonucleotide or the pharmaceutical composition is administered to the subject by intrathecal injection or intracerebroventricular injection. In an embodiment, the cell is a cancer cell. In embodiments of the method, the subject has a cancer selected from breast cancer, colon cancer, brain cancer, astrocytoma, glioma, or neuroblastoma.

In yet another aspect, a method for manipulating splicing of exon 5N or exon 5A of an SCN8A or SCN5A gene transcript is provided, in which the method involves providing an isolated or purified antisense oligonucleotide (ASO), or a pharmaceutically acceptable composition thereof, and allowing the oligonucleotide to bind to a target exon 5N or exon 5A nucleic acid site. In an embodiment, the ASO modifies or alters pre-mRNA splicing of exon 5 in the SCN8A (or SCN5A) sodium ion channel-encoding gene which specifically modulates splicing of the SCN8A or SCN5A exon 5N transcript or which specifically modulates splicing of the SCN8A or SCN5A exon 5A transcript. In an embodiment, the ASO comprises or consists of a sequence selected from ASO-3 of sequence ctgcagaatcaaacca; ASO-8 of sequence gaaagtgcgtagagct; ASO-9 of sequence ctgaaagtgcgtagag; ASO-10 of sequence ccctgaaagtgcgtag; or ASO-12 of sequence agtaccctgaaagtgc. In an embodiment, the ASO modulates pre-mRNA splicing to induce the exclusion (or to decrease the inclusion) of exon 5N or exon 5A of SCN8A RNA. In an embodiment of the method, the antisense oligonucleotide induces an increase in exclusion of exon 5N or of exon 5A of the SCN8A gene. In an embodiment of the above method, an isolated or purified antisense oligonucleotide (ASO) for modifying pre-mRNA splicing of exon 5 in the SCN5A sodium ion channel-encoding gene which specifically modulates splicing of the SCN5A exon 5N or the exon 5A transcript is provided. In an embodiment, the ASO is selected from ASO-11, which comprises or consists of nucleic acid sequence accctgaaagtgcgta or ASO-13, which comprises or consists of nucleic acid sequence ccctcagtaccctgaa. In an embodiment, the ASO is ASO-10, which comprises or consists of nucleic acid sequence ccctgaaagtgcgtag. In an embodiment, the ASO is ASO-24 or ASO-27 as described herein. In an embodiment, the ASO specifically targets, binds, and modulates alternative splicing of SCN5A RNA, thereby specifically decreasing the inclusion of exon 5N or exon 5A of the SCN5A gene. In an embodiment of the method, the antisense oligonucleotide induces a decrease in inclusion of exon 5N or of exon 5A of the SCN5A gene.

In another aspect, a method of altering RNA splicing of exon 5 of a gene encoding a voltage gated sodium ion channel protein in a cell is provided, in which the method involves introducing into the cell an effective amount of the antisense oligonucleotide (ASO) that is capable of altering RNA splicing by inducing a switch from exon 5A to exon 5N expression and transcription. In an embodiment, the ASO is selected from ASO-24, which comprises or consists of nucleic acid sequence atgttctcagcgctga; or ASO-27, which comprises or consists of nucleic acid sequence ggagaaccctgaatgt. In an embodiment, the gene is SCN8A. In an embodiment, the SCN8A gene comprises a missense variant or a gain-of-function mutation or genetic alteration. An embodiment of the method provides an effective amount of the isolated or purified antisense oligonucleotide (ASO) for modifying pre-mRNA splicing of exon 5 in the SCN8A sodium ion channel-encoding gene which specifically modulates splicing of the SCN8A exon 5A transcript. In an embodiment, the ASO is ASO-24, which comprises or consists of the nucleic acid sequence atgttctcagcgctga; or ASO-27, which comprises or consists of the nucleic acid sequence ggagaaccctgaatgt, or a pharmaceutically acceptable composition thereof, introduced into the cell. In an embodiment, the ASOs used in the practice of the method, e.g., ASO-24 or ASO-27, induce a near complete switch from exon 5A to exon 5N in cultured cells and primary neurons without affecting the splicing of other Na⁺ channels or SCN8A mRNA expression. In embodiments, the cells are Sh-SH5Y cells, a human neuroblastoma cell line, or the cells are neuronal cells, primary cortical neurons, neurons, brain cells, or cells of the central nervous system. In embodiments, the cell is in vivo, in vitro, or ex vivo. In an embodiment, the ASO, e.g., ASO-24 or ASO-27, modulates pre-mRNA splicing to induce the exclusion (or to decrease the inclusion) of exon 5A of SCN8A RNA. In an embodiment of the method, the antisense oligonucleotide induces an increase in exclusion of exon 5A of the SCN8A gene. In an embodiment of the above method, an isolated or purified antisense oligonucleotide (ASO) for modifying pre-mRNA splicing of exon 5 in the SCN5A sodium ion channel-encoding gene which specifically modulates splicing of the SCN5A exon 5A transcript is provided. In an embodiment, the ASO includes ASO-24, which comprises or consists of nucleic acid sequence atgttctcagcgctga; or ASO-27, which comprises or consists of nucleic acid sequence ggagaaccctgaatgt. In an embodiment, the ASO specifically targets, binds, and modulates alternative splicing of SCN5A RNA, thereby specifically decreasing the inclusion of exon 5A of the SCN5A gene. In an embodiment, the ASO comprises a modified linkage which is a phosphorothioate linkage. In an embodiment, the ASO comprises a modified sugar moiety which is a 2′-O-methoxyethyl (2′-MOE) group. In an embodiment of the method, the antisense oligonucleotide induces a decrease in inclusion of exon 5A of the SCN5A gene.

In embodiments of any one of the above-delineated methods and/or embodiments thereof, the ASO comprises a modified linkage selected from the group consisting of phosphorothioate, methylphosphonate, phosphodiester, phosphotriester, and phosphorodithioate linkages. In an embodiment, the modified linkage is a phosphorothioate linkage. In an embodiment, the ASO further comprises at least one modified sugar moiety. In embodiments, the modified sugar moiety is a 2′-O-methoxyethyl (2′-MOE) group, a 2′-O-methyl, a 2′-dimethylaminooxyethoxy, a 2′-dimethylaminoethoxyethoxy, a 2′-fluoro, or a 2′-acetamide modification group. In an embodiment, the modified sugar moiety is a 2′-O-methoxyethyl (2′-MOE) group.

In an embodiment of any one of the above-delineated methods and/or embodiments thereof, particularly, a method of treating a disease or disorder, the activity of the voltage-gated sodium ion channel Nav1.6 or Nav1.5 is decreased. In another embodiment of any one of the above-delineated treatment methods and/or embodiments thereof, the ASO may be isolated and/or purified. In embodiments, the isolated and/or purified ASO, or a pharmaceutically acceptable composition thereof, is introduced or administered in combination with another therapeutic agent or drug, and/or wherein one or more antisense oligonucleotides is introduced, administered, or co-administered.

In another aspect, a cell comprising an antisense oligonucleotide of any one of the above-delineated aspects and/or embodiments thereof, and exon 5 of a gene encoding a voltage gated sodium ion channel protein is provided. In an embodiment, the cell comprises an antisense oligonucleotide as described in the above-delineated method and/or embodiments thereof, and exon 5 of a gene encoding a voltage gated sodium ion channel protein. In an embodiment, the gene is SCN8A and the sodium channel protein is Nav1.6. In an embodiment, the gene is SCN5A and the sodium channel protein is Nav1.5. In embodiments, the cell is a neuronal cell, a neuron, a primary cortical neuron, a brain cell, or a cell of the central nervous system. In an embodiment, the cell comprises an isolated or purified antisense oligonucleotide (ASO) for modifying pre-mRNA splicing of exon 5 in the SCN5A sodium ion channel-encoding gene which specifically modulates splicing of the SCN5A exon 5N transcript, and exon 5 of a gene encoding a voltage gated sodium ion channel protein. In an embodiment, the ASO is ASO-11, which comprises or consists of nucleic acid sequence accctgaaagtgcgta; or ASO-13, which comprises or consists of nucleic acid sequence ccctcagtaccctgaa. In an embodiment, the ASO is ASO-10, which comprises or consists of nucleic acid sequence ccctgaaagtgcgtag. In an embodiment, the ASO is selected from an ASO of Table 1 or Table 2. In an embodiment, the ASO specifically targets, binds, and modulates alternative splicing of SCN5A RNA, thereby specifically decreasing the inclusion of exon 5N of the SCN5A gene. In an embodiment, the oligonucleotide comprises a modified linkage which is a phosphorothioate linkage. In an embodiment, the antisense oligonucleotide comprises a modified sugar moiety which is a 2′-O-methoxyethyl (2′-MOE) group. In an embodiment, the cell is a cancer cell. In an embodiment, the cell is contained in, or is derived, obtained, or isolated from a subject having or suspected of having a neurological, neurodevelopmental, or neurodegenerative disorder, disease, or pathology, or a cancer. In an embodiment, the cell is an isolated cell.

In another aspect, a kit (or article or manufacture) including the antisense oligonucleotide of any one of the above-delineated aspects and/or embodiments thereof, packaged in a suitable container, and directions for administering the antisense oligonucleotide to a subject is provided. In an embodiment, the kit includes an isolated or purified antisense oligonucleotide (ASO) for modifying pre-mRNA splicing of exon 5 in the SCN8A sodium ion channel-encoding gene which specifically modulates splicing of the SCN8A exon 5N transcript, or a pharmaceutically acceptable composition thereof. In an embodiment, the ASO comprises or consists of a sequence selected from ASO-3 of sequence ctgcagaatcaaacca; ASO-8 of sequence gaaagtgcgtagagct; ASO-9 of sequence ctgaaagtgcgtagag; ASO-10 of sequence ccctgaaagtgcgtag; or ASO-12 of sequence agtaccctgaaagtgc. In an embodiment, the ASO modulates pre-mRNA splicing to induce the exclusion of exon 5N of SCN8A RNA. In an embodiment, the kit includes an isolated or purified antisense oligonucleotide (ASO) for modifying pre-mRNA splicing of exon 5 in the SCN8A sodium ion channel-encoding gene which specifically modulates splicing of the SCN8A exon 5A transcript, and/or induces a switch from exon 5A to exon 5N in SCN8A, or a pharmaceutically acceptable composition thereof. In an embodiment, the ASO is ASO-24, which comprises or consists of sequence atgttctcagcgctga or ASO-27, which comprises or consists of ggagaaccctgaatgt. In an embodiment, the ASO comprises a modified linkage selected from the group consisting of phosphorothioate, methylphosphonate, phosphodiester, phosphotriester, and phosphorodithioate linkages. In an embodiment, the modified linkage is a phosphorothioate linkage. In an embodiment, the ASO further comprises at least one modified sugar moiety. In embodiments, the modified sugar moiety is a 2′-O-methoxyethyl (2′-MOE) group, a 2′-O-methyl, a 2′-dimethylaminooxyethoxy, a 2′-dimethylaminoethoxyethoxy, a 2′-fluoro, or a 2′-acetamide modification group. In an embodiment, the modified sugar moiety is a 2′-O-methoxyethyl (2′-MOE) group.

Compositions and articles defined by the aspects and embodiments of the present disclosure were isolated or otherwise manufactured in connection with the examples provided below. Other features and advantages of the present disclosure will be apparent from the detailed description, and from the claims.

### Definitions

Unless defined otherwise, all technical and scientific terms used herein have the meaning commonly understood by a person skilled in the art related to the present disclosure. The following references provide one of skill with a general definition of many of the terms used in the present disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In one embodiment, the agent is an inhibitory nucleic acid molecule, such as an antisense oligonucleotide.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, or a 50% or greater change in expression levels.

By “ameliorate” is meant decrease, suppress, attenuate, diminish, arrest, or stabilize the development or progression of a disease, disorder, or pathology. In one embodiment, the disease, disorder, or pathology is a neurological, neurodevelopmental, or neurodegenerative disease, such as epilepsy, epileptic seizures, epileptic encephalopathy, early infantile epileptic encephalopathy, developmental delay, autism, autism spectrum disorder (ASD), mild to severe intellectual disabilities, autism spectrum disorders, movement disorders, developmental and epileptic encephalopathy (DEE), severe myoclonic epilepsy of infancy (SMEI)-borderland (SMEB; also known as borderline SMEI); febrile seizure (FS); epilepsy, generalized, with febrile seizures plus (GEFS+); cryptogenic generalized epilepsy; cryptogenic focal epilepsy; myoclonic-astatic epilepsy; Lennox-Gastaut syndrome; idiopathic spasms; sudden unexpected death in epilepsy (SUDEP); or malignant migrating partial seizures of infancy. In one embodiment, the disease, disorder, or pathology is cancer, e.g., breast cancer, brain cancer, etc., as described herein.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide.

Such biochemical modifications could increase the analog's protease resistance, membrane permeability, or half-life, without altering, for example, ligand binding. An analog may include an unnatural amino acid.

“An antisense oligonucleotide (ASO)” (also called an “antisense oligomer” or “antisense nucleobase oligomer” herein) refers to a molecule that includes a chain of at least eight (8) nucleobases joined together by linkage groups, wherein at least a portion of the oligomer is complementary to a target sequence. Included in this definition are natural and non-natural oligonucleotides, both modified and unmodified, as well as oligonucleotide mimetics such as Protein Nucleic Acids and locked nucleic acids. Numerous nucleobases and linkage groups may be employed in the aspects and embodiments described herein. In an embodiment, the nucleobase oligomer is an antisense oligonucleotide that may contain modified bases, a modified backbone, or any other modification described herein or known in the art. Table 1 provides a list of antisense oligonucleotides (ASOs) directed against the 5N isoform of exon 5 of the voltage gated sodium ion channel protein-encoding gene SCN8A.

TABLE 1
Antisense Oligonucleotides (ASOs)
Targeting SCN8A Exon 5N

| Name | Sequence (5′ to 3′) |
|---|---|
| scn8a_ASO-1 | cttgtaatggtatcac |
| scn8a_ASO-2 | aatggtaccactgggc |
| scn8a_ASO-3 | ctgcagaatcaaacca |
| scn8a_ASO-4 | gcctaggtttacaaac |
| scn8a_ASO-5 | gcgtagagctgaaaca |
| scn8a_ASO-6 | gtgcgtagagctgaaa |
| scn8a_ASO-7 | aagtgcgtagagctga |
| scn8a_ASO-8 | gaaagtgcgtagagct |
| scn8a_ASO-9 | ctgaaagtgcgtagag |
| scn8a_ASO-10 | ccctgaaagtgcgtag |
| scn8a_ASO-11 | accctgaaagtgcgta |
| scn8a_ASO-12 | agtaccctgaaagtgc |
| scn8a_ASO-13 | ccctcagtaccctgaa |
| scn8a_ASO-14 | gggattaccgaaatag |
| scn8a_ASO-15 | ctgggattaccgaaat |
| scn8a_ASO-16 | acccaacacctgacac |
| scn8a_ASO-17 | ctaacccaacacctga |
| scn8a_ASO-18 | gccctaacccaacacc |

Table 2 provides antisense oligonucleotides (ASOs) directed against the 5A isoform of exon 5 of the voltage gated sodium ion channel protein-encoding gene SCN8A.

TABLE 2
Antisense Oligonucleotides (ASOs)
Targeting SCN8A Exon 5A

| Name | Sequence (5′ to 3′) |
|---|---|
| scn8a_ASO-24 | atgttctcagcgctga |
| scn8a_ASO-27 | ggagaaccctgaatgt |

In some embodiments, the oligonucleotide comprises a 2′-O-methoxyethyl (2′-MOE) modification. In some embodiments, the oligonucleotide comprises a gene. In some embodiments, the oligonucleotide comprises a 5-methyluridine. In some embodiments, the oligonucleotide comprises a phosphorothioate linkage between the nucleobases. In some embodiments, the oligonucleotide comprises a phosphorothioate backbone. Phosphorothioate linkages have two stereoisomers (phosphodiester linkages are prochiral). Antisense oligonucleotides (ASOs) that are synthesized by standard methods will comprise diastereomers. The stereoisomers that make up the diastereomers can be isolated, yielding two stereopure oligomer populations. “Stereopure” antisense oligonucleotides comprise a single stereoisomer. (Iwamoto N. et al., 2017, Nat Biotechnol., 35(9):845-851). In some embodiments, the antisense oligonucleotide is a mixture of one or more stereopure molecules with a defined sequence.

In this disclosure, “comprises,” “comprising,” “containing,” and “having” and the like can have the meaning ascribed to them in U.S. Patent law and can mean “includes,” “including,” and the like; “consisting essentially of” or “consists essentially” likewise has the meaning ascribed in U.S. Patent law, and the term is open-ended, allowing for the presence of more than that which is recited so long as basic or novel characteristics of that which is recited is not changed by the presence of more than that which is recited, but excludes prior art embodiments.

“Detect” refers to identifying the presence, absence, or amount of an analyte, agent, or compound to be detected. In one embodiment, a mutation, alteration, or variation in an SCN8A polynucleotide (gene) or encoded polypeptide is detected. In an embodiment, an exclusion (or increase in the exclusion) of an exon 5N isoform of the SCN8A gene is detected. In an embodiment, an inclusion of an exon 5A isoform of the SCN8A gene is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In one embodiment, a disease is a neuronal disease or disorder, i.e., a disease or disorder relating to a nerve cell or a neuron. In embodiments, the disease is a neurological, neurodevelopmental, or neurodegenerative disorder, disease, or pathology, or a symptom thereof; a neuronal disease, or a cancer, that is associated with the splicing of exon 5 and the exon 5 isoforms 5N and/or 5A of the SCN8A or SCN5A genes (i.e., the polynucleotides encoding the Nav1.6 and the Nav1,5 sodium channel proteins, respectively) and/or with the exon 5 isoforms 5N and/or 5A of the SCN8A or SCN5A genes comprising a mutation, or genetic alteration or variation. In one embodiment, a disease is a neurological, neurodevelopmental, or neurodegenerative disorder, disease, or pathology or a symptom thereof, or a cancer, that is associated with the splicing of exon 5 and the exon 5 isoforms 5N and/or 5A of the SCN8A gene and/or with the exon 5 isoforms 5N and/or 5A of the SCN8A gene comprising a mutation, or genetic alteration or variation. In embodiments, neurological, neurodevelopmental, or neurodegenerative disorders, diseases, or pathologies associated with missense variants of SCN8A include, without limitation, epilepsy, epileptic seizures, epileptic encephalopathy, early infantile epileptic encephalopathy, developmental delay, autism, autism spectrum disorder (ASD), mild to severe intellectual disabilities, autism spectrum disorders, movement disorders, developmental and epileptic encephalopathy (DEE), severe myoclonic epilepsy of infancy (SMEI)-borderland (SMEB; also known as borderline SMEI); febrile seizure (FS); epilepsy, generalized, with febrile seizures plus (GEFS+); cryptogenic generalized epilepsy; cryptogenic focal epilepsy; myoclonic-astatic epilepsy; Lennox-Gastaut syndrome; idiopathic spasms; sudden unexpected death in epilepsy (SUDEP); or malignant migrating partial seizures of infancy.

In another embodiment, the disease or disorder is a cancer, or symptom thereof. Nonlimiting examples of cancers include breast cancer, colon cancer, brain cancers, astrocytomas, gliomas, and neuroblastoma. Other cancers are encompassed, such as ovarian cancer, cervical cancer, lung cancer, kidney cancer, prostate cancer, pancreatic cancer, liver cancer, rectal cancer, esophageal cancer, gastrointestinal cancer, testicular cancer, sarcomas, leukemias (B and T cell leukemias), lymphomas, cancers of the immune system, and the like.

By “effective amount” is meant the amount of an agent required to ameliorate the symptoms of a disease relative to an untreated patient. The effective amount of active compound(s) used as described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount of an antisense oligonucleotide is the amount required to alter the splicing of exon 5 and the exon 5 isoforms 5N and/or 5A of the SCN8A or SCN5A genes in a cell or tissue expressing SCN8A or SCN5A exon 5 or an SCN8A or SCN5A exon 5 polynucleotide comprising a mutation, or genetic alteration or variation.

Targets are provided that are useful for the development of highly specific drugs to treat or ameliorate a neurological, neurodegenerative, or neurodevelopmental disorder associated with the methods delineated herein. In addition, the described methods provide a ready means to identify therapies that are safe for use in subjects. In addition, the described methods provide a route for analyzing virtually any number of compounds for effects on a disease described herein with high-volume throughput, high sensitivity, and low complexity.

By “fragment” is meant a portion of a polypeptide or nucleic acid molecule. This portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

A “gain-of-function” mutation, or genetic alteration or variation refers to a mutation, alteration, or variation in a polynucleotide or gene, such as a protein- or polypeptide-encoding polynucleotide or gene, that results in an altered gene product that possesses a new molecular function or a new pattern of gene expression. In an embodiment, a gain-of-function mutation or alteration confers an enhanced or increased activity, or a new activity, on an encoded protein or polypeptide gene product. In an embodiment, a gain-of-function mutation or alteration is a dominant or semi-dominant mutation or alteration. In an embodiment, gain-of-function mutations or alterations in the SCN8A gene encoding the neuronal voltage-gated sodium channel Nav1.6 are associated with early-infantile epileptic encephalopathy type 13 (EIEE13) and intellectual disability. By way of example, in a study of de novo gain-of-function mutations or alterations of SCN8A in patients with intellectual disabilities and epilepsy using electrophysiological analyses, an SCN8A mutant displayed a 10 mV hyperpolarizing shift in voltage dependence of activation (gain of function) in the encoded Nav1.6 gene product. (Blanchard, M. G. et al., 2015, J Med Genet., 52(5):330-337).

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid molecule” is meant a double-stranded or single-stranded oligonucleotide (e.g., siRNA, shRNA, or antisense RNA), or a fragment thereof that when administered to a mammalian cell results in a decrease (e.g., by 10%, 25%, 50%, 75%, or even 90-100%) in the expression of a target gene, target exon, or target isoform of an exon. Typically, an inhibitory nucleic acid comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein. In one embodiment, an antisense oligonucleotide comprises at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more nucleobases complementary to a target sequence, e.g., an exon 5 isoform (5A or 5N) of SCN8A or SCN5A. In embodiments, the antisense oligonucleotide comprises 8-25 nucleobases, 8-20 nucleobases, 8-16 nucleobases, 10-20 nucleobases, 10-19 nucleobases, 10-16 nucleobases, 12-18 nucleobases, or 12-16 nucleobases. In an embodiment, the antisense oligonucleotide comprises 16 nucleobases (a 16-mer). In an embodiment, the antisense oligonucleotide consists essentially of a 16-mer. In an embodiment, the antisense oligonucleotide consists of a 16-mer.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, polynucleotide, polypeptide, or peptide as described is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule (e.g., DNA, RNA) that is free of the genes or polynucleotides that flank the gene or polynucleotide in the naturally-occurring genome of the organism from which the nucleic acid molecule is derived or obtained. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide that has been separated from components that naturally accompany it. Typically, the polypeptide is isolated when it is at least 60%, by weight, free from the proteins and naturally-occurring organic molecules with which it is naturally associated. In some embodiments, a preparation contains at least 75%, at least 85%, at least 90%, at least 95%, or at least 99%, by weight, of the isolated polypeptide of interest. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level, activity, or structure that is associated with a disease or disorder.

As used herein, “obtaining” as in “obtaining” an agent or sample, includes synthesizing, purchasing, isolating, purifying, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition. In an embodiment, a reference refers to a voltage gated sodium ion channel encoding gene or polypeptide in an untreated control cell, or the expression of a voltage gated sodium ion channel gene or polynucleotide in an untreated control cell. In an embodiment, a reference refers to a voltage gated sodium channel encoding gene or polypeptide in an untreated control cell, or the expression of a voltage gated sodium ion channel exon 5 gene or polynucleotide, or exon 5A or 5N gene or polynucleotide in an untreated control cell. In an embodiment, a reference refers to a voltage gated sodium ion channel encoding gene or polypeptide comprising a mutation or alteration associated with an SCN8A (or SCN5A) disease, disorder, or pathology in an untreated control cell, or the expression of a voltage gated sodium ion channel exon 5 gene or polynucleotide, or exon 5A or 5N gene or polynucleotide comprising a mutation or alteration associated with an SCN8A (or SCN5A) disease, disorder, or pathology in an untreated control cell. In another embodiment, a reference refers to the expression of an SCN8A (or SCN5A) polynucleotide, or exon 5N or 5A SCN8A (or SCN5A) polynucleotide or sodium ion channel gene product thereof in a cell expressing a wild-type SCN8A (or SCN5A) encoded polypeptide or polynucleotide.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 10, 15, or 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 5, 10, 15, 20, or 50 nucleotides, at least about 60 nucleotides, at least about 75 nucleotides, at least about 100 nucleotides, or at least about 300 nucleotides or any integer thereabout or therebetween.

“RNA splicing” refers to a process in molecular biology in which a newly-made precursor messenger RNA (pre-mRNA) transcript is transformed into a mature messenger RNA (mRNA). RNA splicing functions by removing (or excising) introns (non-coding regions of RNA) out of the primary messenger RNA transcript (pre-mRNA) and splicing (joining) back together exons (coding regions of proteins) to generate mature mRNA, which is translated into protein. For nuclear-encoded genes in eukaryotic cells, splicing occurs in the nucleus either during, or immediately after, transcription. For those eukaryotic genes that contain introns, splicing is usually needed to create a mature mRNA molecule that can be translated into protein. For many eukaryotic introns, splicing occurs in a series of reactions that are catalyzed by the spliceosome, a complex of small nuclear ribonucleoproteins (snRNPs). There exist self-splicing introns, i.e., ribozymes that can catalyze their own excision from their parent RNA molecule. The process of mRNA transcription, splicing and translation is called gene expression, the central dogma of molecular biology. Alternative splicing refers to the process of selecting or utilizing different combinations of splice sites within a messenger RNA precursor (pre-mRNA) to produce variably spliced mRNAs, e.g., exon 5N and exon 5A mRNAs. The multiple distinct functional mRNA transcripts, which are produced from a single gene that is alternatively spliced, can encode distinct proteins that vary in their amino acid sequence and activity.

SCN8A exon 5N refers to a polynucleotide sequence having at least 85%, at least 90%, at least 95%, at least 98%, at least 99%, or complete nucleic acid sequence identity to the human SCN8A (5N) sequence (GenBank Accession No.: NG 021180.3) shown below:

5N:
gtatataacagagtttgtaaacctaggcaatgtttcagctctacgcact
ttcagggtactgagggctttgaaaactatttcggtaatcccag

SCN8A exon 5A refers to a polynucleotide sequence having at least 85%, at least 90%, at least 95%, at least 98%, at least 99%, or complete nucleic acid sequence identity to the human SCN8A (5A) sequence shown below:

5A:
atatgtgacagagtttgtggacctgggcaatgtctcagcgctgagaaca
ttcagggttctccgagctttgaaaactatctctgtaattccag

By “specifically binds” is meant a polynucleotide that recognizes and binds a complementary sequence of a polynucleotide, e.g., an SCN8A (or SCN5A) exon 5, exon 5A and/or exon 5N as described herein. An antisense oligonucleotide (ASO) may specifically bind its perfect complement or a sequence to which it is not perfectly complementary. In one embodiment, the antisense oligonucleotide specifically binds a polynucleotide that comprises 1, 2, 3, 4, 5 or more bases that are not perfectly complementary to the antisense oligonucleotide.

Nucleic acid molecules useful in the described methods include any nucleic acid molecule that encodes a polypeptide, e.g., a sodium ion channel polypeptide, or a functional fragment or portion thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the described methods include any nucleic acid or polynucleotide molecule or gene that encodes a polypeptide as described, or a functional fragment or portion thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, 1987, Methods Enzymol. 152:399; Kimmel, A. R., 1987, Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an: embodiment, hybridization will occur at 30° C. in 750 mM NaCl, 75 mM trisodium citrate, and 1% SDS. In an embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, in some embodiments, stringent salt concentration for the wash steps will be less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80%, 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a non-human primate, bovine, equine, canine, ovine, or feline.

As used herein, the terms “treat,” treating,” “treatment,” and the like refer to reducing or ameliorating a disease and/or symptoms associated therewith. It will be appreciated, although not precluded, that treating a disease, disorder, or condition does not require that the disease, disorder, condition, or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B-1, 1B-2, and 1B-3 provide illustrations that depict the assay developed to determine the ration of exons 5A and 5N of SCN8A and structural depictions of exon 5 of SCN8A. FIG. 1A illustrates the steps involved in an assay designed to determine the ratio of SCN8A exons 5A and 5N (a 5A:5N ratio assay). As shown in FIG. 1A, isolated RNA from cultured ND7/23 cells or brain tissue was isolated using the Zymo Quick-RNA kit. RNA was subjected to RT-PCR targeting exon 4 and exon 6 for primer binding. Following RT-PCR, the PCR product was digested using enzymes targeting either the 5A (AvaII) exon 5 isoform of SCN8A or the 5N (StyI) exon 5 isoform of SCN8A. The digested as well as the undigested PCR products were then electrophoresed on an agarose gel for quantification. The gel depiction in the lower left of FIG. 1A shows the digestion results that can be obtained. On the left side of the gel, the first three lanes (“undigested,” “5A digest (AvaII)”, “5N digest (StyI or XmaJI)”) show the results if expression of the exon 5 isoform in cells/tissue is exclusively that of isoform 5A. Accordingly, when digestion by the AvaII restriction enzyme (which targets and cleaves the 5A isoform of SCN8A) occurs, the entire PCR product is digested as evidenced by the lower-sized band in the “5A Digest (AvaII)” lane of the gel. In contrast, when the StyI (the 5N isoform targeting enzyme) is used, no digestion occurs, as evidenced by the higher band in the “5N digest (StyI or XmaJI)” lane of the gel, which migrates at the same position as the “undigested” product. The three middle lanes of the gel show the results if expression of the exon 5 isoform of SCN8A in cells/tissue is exclusively that of isoform 5N. As seen in lane 3 of the middle portion of the gel, digestion occurs only when a 5N targeting enzyme such as StyI is used, as evidenced by the smaller-sized (lower) band. When the restriction enzyme AvaII that targets the 5A isoform of exon 5 of SCN8A is used, no digestion is observed (the AvaII band migrates at the same position as the “undigested” sample). The last three lanes in the rightmost portion of the gel depict the results if the cells/tissue express a 1:1 ratio of the 5A:5N isoforms of exon 5 of SCN8A. In this case, equal bands of both digested and undigested products are shown following the use of restriction enzymes that target the 5A isoform and the 5N isoform, demonstrating 1:1 5A:5N isoform expression. FIGS. 1B-1 depicts exon 5 of SCN8A (Epilepsy Genetics Initiative, 2018, Genet Med, 20(2):275-281). FIG. 1B-1 (i) illustrates that SCN8A exon 5 includes two sequences, exons 5N and 5A) in the genome. Only one of the two exons remains in the transcripts after splicing occurs. In FIG. 1B-2, the nucleotide (ii) and amino acid (iii) sequences of exons 5N and 5A are nearly identical; asterisks indicate the sites having the same sequence and arrows indicate the sites at which a disease-causing variant was identified in exon 5A. All disease-causing variants were identified at sites where the sequence was identical between exons 5A and 5N (Epilepsy Genetics Initiative, Ibid.) The two upward arrows in (iv) of FIG. 1B-3 show exon 5 relative to the Nav1.6 protein, which spans two transmembrane domains and a small extracellular region of Nav1.6. (Plummer N. W. et al., 1997, J Biol Chem, 272:24008-24015). As shown in FIG. 1B-3, SCN8A contains two pairs of tandemly duplicated, mutually exclusive, alternatively spliced exons, i.e., exons 5A/5 N and exons 18 A/18 N, with a common evolutionary origin. Exon 5 encodes portions of transmembrane segments S3 and S4 in domain I of the voltage gated sodium ion channel Nav1.6. Exons 5 N (neonatal) and 5 A (adult) differ by two out of 31 amino acids (FIG. 1B-2, (iii)). In humans and rodents, there is evidence that the expression of exon 5 A increases during development; the expression of “neonatal” exon 5 N decreases over time, but it continues to be expressed at a low level in the adult brain. Exon 18 encodes portions of transmembrane segments S3 and S4 in domain III of Nav1.6. The 18 N transcript contains a conserved in-frame stop codon, which is predicted to result in protein truncation and is widely expressed at low levels in nonneuronal tissue.

FIGS. 2A-2C provide images of agarose gels and a graphic quantification of the gels related to the 5A:5N ratio assay of ND7/23 ASO treated cells as described in Example 2. FIG. 2A: Agarose gel of isolated RNA from ASO treated ND7/23 cells following RT-PCR and StyI digestion (StyI: 5N isoform targeting digest). Markers are run in the lane labeled “ASO” in the gel (labeled 350 bp, 300 bp and 250 bp). The first lane of the gel next to the marker lane and designated by a black dot, contains a sample from untreated (undigested) ND7/23 cells. The following lanes are ND7/23 samples treated with different ASOs, numbered as shown in Table 1, (500 nM), and run in triplicate. FIG. 2B: Agarose gel of isolated RNA from ASO treated ND7/23 cells following RT-PCR and AvaII digestion (AvaII: 5A isoform targeting digest). The first lane of the gel next to the marker lane (labeled as described for FIG. 2A) contains a sample form untreated ND7/23 cells. The following lanes are ND7/23 samples treated with different ASOs, numbered as shown in Table 1, (500 nM), and run in triplicate. FIG. 2C: Quantification of the results of the agarose gels shown in FIGS. 2A and 2B. The SCN8A 5A:5N ratio was determined using ImageJ, analyzing the band area of the digested and undigested samples; dividing those values by the total band area allows for determination of percent digested.

FIG. 3 presents bar graphs demonstrating the relative inclusion of exon 5N of different sodium ion channel genes (“SCNXA”) in ND7/23 cells treated or untreated (control) with the ASOs as shown on the X-axis (“Condition”) and described herein (Example 4). For each condition shown in the graph, the first bar (leftmost) reflects that primers specific for isolated SCN2A cDNA (small circle) were used; the second bar from the left reflects that primers specific for isolated SCN3A cDNA (small square) were used; the third bar from the left reflects that primers specific for isolated SCN5A cDNA (small triangle) were used; and the fourth bar from the left reflects that primers specific for isolated SCN9A cDNA (small upside-down triangle) were used.

FIGS. 4A-4D show via RNA gels and bar graphs the results of experiments conducted to identify exon 5N to exon 5A splice-switching ASOs. The effect of ASOs (1000 nM) in ND7/23 cells after culturing the cells in the presence of the ASOs for 24 hours is shown in FIG. 4A. The effect of ASOs (1000 nM) in primary cortical neurons from P1 mice after culturing the cells in the presence of the ASOs for 24 hours is shown in FIG. 4B. FIG. 4C shows the effect of ASOs on exon 5N inclusion in SCN2A and SCN3A. As observed, the tested ASOs (ASO-10, ASO-11, and ASO-13) had minimal effect on exon 5N inclusion in SCN2A and SCN3A. FIG. 4D shows the effect of the ASOs on SCN8A mRNA levels as determined by qPCR. As observed, the tested ASOs (ASO-10, ASO-11, and ASO-13) had minimal effect on SCN8A mRNA levels. The data show mean±SEM from n=3-6 replicates.

FIGS. 5A-5D show via RNA gels and bar graphs the results of experiments conducted to identify 5A to 5N splice-switching ASOs in neurons/neuron cells. The effect of ASOs (1000 nM) in Sh-SH5Y cells (FIG. 5A) and in primary cortical from P1 mice (FIG. 5B) after 24 hours is shown. FIG. 5C shows the effect of ASOs on exon 5N inclusion in SCN2A and SCN3A, and FIG. 5D shows the effect of ASOs on SCN8A mRNA levels as determined by qPCR. The results demonstrate that the 5A to 5N splice-switching ASOs, ASO-24 and ASO-27, had relatively minimal effect on exon 5N inclusion in SCN2A and SCN3A, and did not affect SCN8A mRNA levels. The data show mean±SEM from n=3-6 replicates.

FIGS. 6A-6D provide an experimental schematic, bar graphs and line graphs related to experiments conducted to investigate the efficacy and safety of 5N targeting ASOs in vivo. FIG. 6A: P1 C57BL/6 pups were intracerebroventricularly (ICV) injected with 30 μg of ASO control (CTL), or ASO-13, or were untreated. At P7, developmental assays as known in the art were conducted on pups to determine normal development, such as daily weighing, negative geotaxis, and righting reflex assays. Fourteen days post-injection, the animals were euthanized and their brains were isolated for RNA analysis. RNA was isolated from the brains and was converted to cDNA followed by PCR. FIG. 6B: PCR products were digested with restriction enzymes StyI (5N specific) and AvaII (5A specific). The resulting products were then electrophoresed on a 3% agarose gel and quantified to determine percent SCN8A 5A:5N using ImageJ. The pups' weights were tracked from P7-P14, as shown in FIG. 6C. Negative geotaxis assays were conducted to determine the time taken by a pup to make a 180° turn on a inclined ramp. The results of these assays using ASO-treated (e.g., ASO-13-treated) and untreated pups from P7-P14 are shown in FIG. 6D.

### DETAILED DESCRIPTION

Described and featured herein are antisense oligonucleotides (ASOs) that alter the splicing of exon 5, e.g., exon 5N, of a voltage-gated sodium ion channel-encoding gene. Mutations or genetic alterations in voltage-gated sodium channels have been implicated in a number of neuronal. neurological, neurodevelopmental, or neurodegenerative disorders, diseases, or pathologies, and in cancers. The activity of voltage-gated sodium ion channels has long been associated with disorders and diseases of neuronal (nerve cell) excitability, for example, epilepsy and chronic pain. More recently, the role of sodium channels in health and disease has been expanded to include autism, migraine, multiple sclerosis, cancer, as well as muscle and immune system disorders.

In embodiments, the voltage-gated sodium ion channel-encoding genes include SCN1A, SCN2A, SCN3A, SCN5A, SCN8A, and SCN9A. In an embodiment, the voltage-gated sodium ion channel gene is SCN8A, which encodes the voltage-gated sodium ion channel Nav1.6 The terms “sodium channel” and “sodium ion channel” are used interchangeably herein.

Exon 5 of sodium ion channel-encoding genes such as SCN8A exists in two versions or isoforms called exon 5N and exon 5A. Exons 5N and 5A are alternatively spliced exons that encode transmembrane segments of domain I of the SCN8A gene. The 5N and 5A exon isoforms encode sodium channels that differ by a single amino acid at position 7. The aspects and embodiments described herein are based, at least in part, on the discovery of antisense oligonucleotides (ASOs) that change the splicing of exon 5 of voltage-gated sodium channels. In an embodiment, ASOs identified and described herein switch exon 5 of SCN8A from the 5N isoform to the 5A isoform. As such, the compositions and methods involving the ASOs provided herein can be used to significantly impact the splicing of SCN8A exon 5, e.g., in a cell or tissue, by inducing or promoting the exclusion of exon 5N, thereby reducing exon 5N inclusion by at least 40% or greater in the cell or tissue. In an embodiment, ASOs identified and described herein switch exon 5 of SCN8A from the 5A isoform to the 5N isoform. Thus, the compositions and methods involving the ASOs provided herein can be used to significantly impact the splicing of SCN8A exon 5, e.g., in a cell or tissue, by inducing or promoting the exclusion of exon 5A, thereby reducing exon 5A inclusion (and/or increasing exon 5N inclusion) in the cell or tissue. In an embodiment, the designed and generated ASOs as described herein were found to be specific to the sodium ion channel-encoding gene SCN8A. Such SCN8A-specific ASOs were found to induce little to no change in exon 5N inclusion of SCN2A, SCN3A, SCN5A or SCN9A.

Because mutations or alterations in exon 5 of SCN8A may be associated with neurological, neurodevelopmental, or neurodegenerative disorders, diseases, or pathologies, such as epilepsy, epileptic encephalopathy, and fetal and infantile seizures (Arisaka, A. et al., 2021, Epilepsy Behav Rep, 15:100417; Ohba, C. et al., 2014, Epilepsia, 55(7):994-1000; Pergande, M. et al., 2020, Genet Med, 22(3):511-523; Hu, C., T. Luo and Y. Wang, 2022, Seizure, 95:38-49; McNally, M. A. et al., 2016, Pediatr Neurol., 64:87-91; Ademuwagun, I. A. et al., 2021, Front Neurol., 12:600050; and Wang, J. et al., 2017, BMC Med Genet, 18(1):104), modulating splicing of the SCN8A gene product to the alternative exon isoform could allow for recovered function of SCN8A and reduce seizure occurrence in patients with mutations or alterations in exon 5.

In an embodiment, ASOs designed to be specific for exon 5 of SCN8A encoding the Nav1.6 sodium ion channel as described herein (e.g., Table 1), were found to cause a switch from exon 5N to exon 5A of SCN8A. In an embodiment as described and exemplified herein, ASOs 3 and ASOs 5-13 shown in Table 1 all impacted the splicing of SCN8A exon 5 and induced or promoted the exclusion of exon 5N transcription. The inclusion of transcription of exon 5N decreased from 83% to an average of 16%. In an embodiment, the ASOs of Table 1 may be useful to target the mRNA associated with exon 5 of other sodium ion channel-encoding genes, such as, without limitation, SCN5A. In an embodiment, methods of using such ASOs are described. In an embodiment, ASO-3 of sequence ctgcagaatcaaacca; ASO-8 of sequence gaaagtgcgtagagct; ASO-9 of sequence ctgaaagtgcgtagag; AS)-10 of sequence ccctgaaagtgcgtag; and ASO-12 of sequence agtaccctgaaagtgc, as set forth in Table 1, specifically target, bind, and modulate alternative splicing of SCN8A RNA, thereby specifically decreasing the inclusion of exon 5N of the SCN8A gene. These ASOs do not induce a decrease in exon 5N expression in other sodium channel genes, e.g., SCN2A, SCN3A, SCN5A, and SCN9A, in which alternative splicing of exon 5 occurs to generate the exon 5N and 5A isoforms. In another embodiment, ASOs designed to be specific for exon 5 of SCN8A encoding the Nav1.6 sodium ion channel as described herein (e.g., Tables 2 and 3), e.g., ASO-24 of sequence atgttctcagcgctga and ASO-27 of sequence ggagaaccctgaatgt, were found to cause a switch from exon 5A to exon 5N of SCN8A. The ASOs identified and provided herein are advantageous for use in therapeutic treatment of SCN8A-associated diseases and disorders, because there are currently no treatments that specifically target the SCN8A gene and its encoded Nav1.6 protein and no disease-modifying agents that target the activity of SCN8A or SCN8A exon 5.

### Voltage-Gated Sodium Channels

Sodium channels (or sodium ion channels) are large integral membrane proteins that are encoded by at least ten genes in mammals and form ion channels that conduct sodium ions (Na⁺) through the plasma membrane of a cell. Selective permeation of sodium ions through voltage-dependent sodium channels is fundamental to the generation of action potentials in excitable cells such as neurons. The different sodium channels have remarkably similar functional properties; however, small changes in sodium channel function are biologically relevant, and mutations or alterations in the sodium channels cause several human diseases of hyperexcitability. (F. Y. Yu et al., 2003, Genome Biology, Vol. 4, No. 207: 207.1-207.7).

Voltage-gated sodium channels play an essential role in the initiation and propagation of action potentials in neurons and in other electrically excitable cells, such as myocytes and endocrine cells. When the cell membrane is depolarized by a few millivolts, sodium channels activate and inactivate within milliseconds. The influx of sodium ions through the integral membrane proteins comprising the cell's ion channel further depolarizes the membrane and initiates the rising phase of the action potential. A voltage-gated sodium channel is a large, multimeric complex that is composed of an alpha (a) subunit and one or more smaller beta (β) subunits. The ion-conducting aqueous pore is contained entirely within the a subunit. Indeed, the essential elements of sodium-channel function, namely, channel opening, ion selectivity, and rapid inactivation, can be demonstrated when a subunits are expressed alone in heterologous cells. Co-expression of the B subunit is required for the full reconstitution of the properties of native sodium channels, as these auxiliary subunits modify the kinetics and voltage-dependence of the opening and closing (gating) of the channel. Although different sodium channels have broadly similar functional characteristics, small differences in properties can distinguish different isoforms and contribute to their specialized functional roles in mammalian physiology and pharmacology. (F. Y. Yu et al., 2003, Genome Biology, Vol. 4, No. 207: 207.1-207.7)

### Voltage-Gated Sodium Channel Isoforms and Isoform Expression

Genetic variants in the genes SCN1A, SCN2A, SCN3A, and SCN8A are leading causes of epilepsy, epileptic encephalopathy, developmental delay, and autism spectrum disorder (ASD). These four homologous genes encode voltage-gated sodium ion channels, i.e., Nav1.1, Nav1.2, Nav1.3, and Nav1.6, respectively, which are critical for a range of functions in the central nervous system, including axonal action potential initiation and propagation, dendritic excitability, macroscopic anatomical development, and activity-dependent myelination (See, Liang, L. et al., 2021, Genome Medicine, 13, No. 135 (doi.org/10.1186/s13073-021-00949-0).

The mRNA splicing patterns of all four of these genes vary across development in the rodent brain, including mutually exclusive copies of the fifth protein-coding exon detected in the neonate (5N) and adult (5A). A second pair of mutually exclusive exons has been reported in SCN8A only (18N and 18A). The expression of individual exons has been quantified in the developing human brain. (See, Liang, L. et al., 2021, Genome Medicine, 13, No. 135 (doi.org/10.1186/s13073-021-00949-0).

The multiple exons that constitute the sodium channel genes can be protein-coding sequences (CDS), untranslated regions (UTRs), or non-coding exons (NCEs). Gene isoforms are differing combinations of these exons, which can change the amino acid sequence of the encoded proteins (protein isoforms or proteoforms). A well-characterized isoform change across these four sodium channels involves the two, mutually exclusive copies of the fifth protein-coding exon. This exon encodes part of the first domain (domain I) of the Nav channel, including the end of transmembrane segment S3, most of transmembrane segment S4, aid a short extracellular linker connecting these two segments. (Liang, L. et al., Ibid.). In humans, each copy of this fifth protein-coding exon is 92 nucleotides in length, encoding 30 amino acids, of which one to three amino acids vary between the two exon copies for each gene. “A” isoforms of exon 5 include the ancestral and canonical copy of exon 5 (5A), with an aspartic acid residue (Asp/D) encoded at position 7 of 30. “N” isoforms of exon 5 use the alternative copy of exon 5 (5N), with an asparagine residue (Asn/N) at position 7 of 30 in SCN1A, SCN2A, and SCN8A, and a serine residue (Ser/S) in SCN3A. (Liang, L. et al., Ibid.). While this reflects a relatively small change in protein structure, the differential inclusion of exon 5A or exon 5N can have distinct effects on channel function. For example, these splice isoforms can alter channel electrophysiological characteristics, the functional impacts of variants associated with seizure, neuronal excitability, response to anti-epileptic drugs/therapeutics, and seizure-susceptibility. (Liang, L. et al., Ibid).

The utilization of exon 5A or exon 5N varies across developmental periods in mammals, with exon 5N generally being expressed at higher levels in the neonatal period and exon 5A being predominant in adults. This switch is defined best in mouse, where the 5A:5N ratio varies by gene and brain region along with developmental stage. For SCN21A in mouse neocortex, the 5A:5N ratio is 1:2 at birth (postnatal day 0/P0) and changes to 3:1 by P15. For both SCN3A and SCN8A, exon 5A predominates throughout the postnatal period, with a 2:1 ratio at P0 increasing to 5:1 by P15. SCN1A lacks a functional copy of exon 5N in the mouse genome. Similar developmental profiles have not been reported for humans beyond the 5A/5N utilization in SCN1A in adults, in which a 5A:5N ratio of over 5:1 was observed in the temporal cortex and hippocampus of adult surgical resections. (Liang, L. et al., Ibid.).

In addition to the 5A/5N switch, a similar developmental shift in mutually exclusive exons has been reported for exons 18A or 18N in SCN8A, which is regulated by the RNA-binding protein RBFOX1. In the embryonic mouse brain, most SCN8A transcripts include exon 18N or skip both exons 18N and 18A, leading to non-functional channels, while exon 18A predominates in the adult mouse and human brain. (Liang, L. et al., Ibid.). The utilization of protein-coding exons in four seizure-associated voltage-gated sodium channels in the human and mouse neocortex across development was assessed by Liang, L. et al, Ibid., A synchronized transition from exon 5N to exon 5A utilization was found between 24 post-conceptual weeks (2nd trimester) and six years of age across all four voltage-gated sodium channels, and a transition from exon 18N to exon 18A in SCN8A was found from 13 post-conceptual weeks to 6 months of age.

Because such isoform differences can modify the function of the encoded voltage-gated sodium channels, the ASOs described herein can provide beneficial and advantageous interventions for use in manipulating and modifying the SCN8A exon 5 isoform ratio as a potential therapy for disorders caused by variants in the SCN8A sodium channel gene and/or without limitation, for neurological, neurodevelopmental, or neurodegenerative disorders, diseases, or pathologies such as epilepsy, epileptic encephalopathy, developmental delay, and autism spectrum disorder (ASD). In an embodiment, the ASOs described herein can specifically target differential splicing patterns and provide therapeutic intervention for manipulating the expression of specific exons, namely, exon 5N and 5A, to treat diseases and disorders associated with differential exon utilization and expression, including variations and mutations or alterations in the exons. The ASOs provided herein can be used alone or in combination with existing therapies, for example, anti-epileptic drugs and the like, in treatment methods. In an embodiment, the treatments are administered intrathecally.

### SCN8A Encoding Sodium Ion Channel Nav1.6

Sodium channel protein type 8 subunit alpha (also known as Nav1.6) is a membrane protein that is a member of the voltage-gated sodium channel alpha subunit gene family and is encoded by the SCN8A gene. Sodium channels allow positively charged sodium (Na) atoms (sodium ions) to pass into cells, for example, neurons and neuronal cells; they play a key role in a cell's ability to generate and transmit electrical signals. The encoded Nav1.6 protein forms the ion pore region of the voltage-gated sodium channel. This protein is essential for the rapid membrane depolarization that occurs during the formation of the action potential in excitable neurons. Mutations or alterations in the SCN8A gene are associated with cognitive disability, pancerebellar atrophy, and ataxia. Alternate splicing results in multiple transcript variants.

SCN8A is associated with inherited neurological disorders in the mouse that include ataxia, dystonia, severe muscle weakness, and paralysis. The complete coding sequence and exon organization of the human SCN8A gene was reported by Plummer, N. W. et al., 1998, Genomics, 54(2):287-296. The predicted 1980 amino acid residues are distributed among 28 exons, including two pairs of alternatively spliced exons. The protein encoded by the SCN8A gene is evolutionarily conserved (98.5% amino acid sequence identity between human and mouse). Consensus sites for phosphorylation of serine/threonine and tyrosine residues are present in cytoplasmic loop domains. The polymorphic (CA)ₙ microsatellite marker D12S2211, with PIC=0.68, was isolated from intron 10C of SCN8A. Single nucleotide polymorphisms in intron 19 and exon 22 were also identified. SCN8A was localized to chromosome band 12q13.1 by physical mapping on a YAC contig. (Plummer et al., 1998, Ibid.)

Nav1.6 channels are primarily found in the nerve cells (neurons) of the brain and spinal cord (central nervous system or CNS) and in neurons that connect the CNS to muscles and sensory cells of the peripheral nervous system. Nav1.6 channels control the flow of sodium ions into cells, which makes it possible for neurons to communicate by generating and transmitting electrical signals.

SCN8A Related Epilepsy with Encephalopathy

More than 100 mutations or genetic alterations in the SCN8A gene have been found to cause SCN8A-related epilepsy with encephalopathy, a condition characterized by recurrent seizures (epilepsy), abnormal brain function (encephalopathy), impaired speech and motor function, and intellectual disability. The signs and symptoms of this condition typically begin in infancy. Typically, SCN8A encephalopathy patients have multiple seizure types, and onset is generally not associated with fever or illness. Most of these SCN8A gene mutations or alterations result in a change in a single amino acid in the Nav1.6 sodium ion channel. The mutations or alterations that cause SCN8A-related epilepsy with encephalopathy result in altered channels that remain open longer than channels encoded by normal, nonmutated SCN8A, which increases the flow of sodium ions into neurons. The persistently open channels increase electrical signals in an abnormal fashion, which can lead to excess activation (excitation) of neurons in the brain. Such increased neuronal activity leads to seizures in individuals with SCN8A-related epilepsy with encephalopathy.

Three disease-causing variants in exon 5A of the SCN8A gene and five disease-causing variants in exon 5N of the SCN8A gene, which are associated with epilepsy, have been reported. (Epilepsy Genetics Initiative, 2018, Genet Med, 20(2):275-281).

The seizures in SCN8A-related epilepsy with encephalopathy include involuntary muscle contractions that occur before the age of 1 year (infantile spasms), partial or complete loss of consciousness (absence seizures), involuntary muscle twitches (myoclonic seizures), or loss of consciousness with muscle rigidity and convulsions (tonic-clonic seizures). Most individuals having SCN8A-related epilepsy with encephalopathy have more than one type of seizure. The frequency of seizures in different individuals with this condition ranges from hundreds per day to fewer than one per month. In many individuals, the seizures are described as refractory because they do not respond to therapy with anti-epileptic medications.

Other signs and symptoms of SCN8A-related epilepsy with encephalopathy include intellectual disability that may be mild to severe. Some affected infants have normal early development but begin to lose previously acquired skills (developmental regression) and have a gradual loss in thinking ability (cognitive decline) when epilepsy develops and after the onset of seizures. Problems with movement are common, and about half of affected infants cannot perform intentional movements. Behavior disorders may also occur. In rare cases, individuals with this condition die unexpectedly for no known reason (sudden unexpected death in epilepsy or SUDEP). Without limitation, SCN8A expression is primarily found in brain, adrenal gland, gall bladder, and testis.

### Other Sodium Ion Channel-Encoding Genes

### SCN3A and SCN4A (Voltage-Gated Sodium Channel Alpha Subunits 3 and 4)

SCN3A and SCN4A encode voltage-gated sodium channels, which are transmembrane glycoprotein complexes composed of a large alpha subunit with 24 transmembrane domains and one or more regulatory beta subunits whose activity generates and propagates action potentials in neurons and muscle.

The SCN3A gene encodes one member of the sodium channel alpha subunit gene family, and is found in a cluster of five alpha subunit genes on chromosome 2. Multiple transcript variants encoding different isoforms have been found for the SCN3A gene, which exhibits biased expression in brain, adrenal gland, appendix, lymph node, fat, gall bladder, small intestine, colon, spleen, stomach, testis, thyroid, and urinary tract/bladder.

Defects in the SCN4A gene cause paramyotonia congenita of von Eulenburg (PMC) [MIM:168300], an autosomal-dominant sodium channel disease characterized by muscle stiffness due to membrane hyperexcitability. Missense mutation causes small inward current (smaller than 3%) that result in enhanced excitability (myotonic stiffness). In addition, defects in SCN4A are a cause of paramyotonia without cold paralysis [MIM:168350], an autosomal-dominant disease characterized by myotonia not increased by cold exposure. Defects in SCN4A are a cause of hypokalemic periodic paralysis (HYPOKPP or HOKPP) [MIM:170400], which is an autosomal-dominant sodium channel disorder characterized by episodic muscle weakness associated with low-serum potassium. Muscle weakness may be caused by altered excitation-contraction coupling in I-HYPOKPP patients. Defects in SCN4A may also be a cause of hyperkalemic periodic paralysis (HYPP) [MIM: 170500], an autosomal-dominant sodium channel disease characterized by episodic attacks of muscle weakness. While attacks of muscle weakness can be provoked in HYPP patients by oral potassium administration, this is a remedy for HYPOKPP.

### SCN5A (Sodium Voltage-Gated Channel Alpha Subunit 5)

The sodium ion channel encoded by the SCN5A gene is an integral membrane protein and tetrodotoxin-resistant voltage-gated sodium channel subunit, which is found primarily in cardiac muscle and is responsible for the initial upstroke of the action potential in an electrocardiogram. Defects in the SCN5A gene have been associated with long QT syndrome type 3 (LQT3), atrial fibrillation, cardiomyopathy, and Brugada syndrome, all of which are autosomal dominant cardiac diseases. Alternative splicing results in several transcript variants encoding different isoforms.

### SCN2A (Sodium Voltage-Gated Channel Alpha Subunit 2)

The SCN2A gene encodes one member of the sodium channel alpha subunit gene family. Allelic variants of this gene are associated with seizure disorders and autism spectrum disorder. Alternative splicing results in multiple transcript variants. SCN2A is expressed primarily in the brain and kidney.

### SCN1A (Sodium Voltage-Gated Channel Alpha Subunit 1)

The SCN1A gene encodes a sodium channel alpha subunit, which has four homologous domains, each of which contains six transmembrane regions. Allelic variants of this gene are associated with generalized epilepsy with febrile seizures and epileptic encephalopathy. Alternative splicing results in multiple transcript variants. SCN1A is expressed primarily in the brain, lung, and testis.

### Neurological, Neurodevelopmental, or Neurodegenerative Disorders, Diseases, or Pathologies

### Epilepsy

Epilepsy is a neurological disorder that currently affects about 3.4 million people nationwide and is characterized by the recurrence of seizures (Zack, M. M. et al., 2017, Morbidity and Mortality Weekly Report (MMWR), 66:821-825). Mutations or alterations in SCN8A, which encodes the Nav1.6 sodium ion channel and is crucial in the generation and regulation of action potentials in neurons, are implicated in neurological, neurodegenerative, and neurodevelopmental diseases and disorders, such as epilepsy, etc., as well as mild to severe intellectual disabilities. Examples of such intellectual disabilities include, without limitation, autism spectrum disorders, movement disorders, developmental and epileptic encephalopathy (DEE), seizures and Sudden Unexpected Death in Epilepsy (SUDEP). SCN8A-related epilepsy and/or neurodevelopmental disorders encompass a spectrum of phenotypes. Epilepsy phenotypes include developmental and epileptic encephalopathy (DEE) associated with severe developmental delays and usually pharmaco-resistant epilepsy with multiple seizure types; mild-to-moderate developmental and epileptic encephalopathy (mild/modDEE, or intermediate epilepsy) with partially treatable epilepsy; self-limited familial infantile epilepsy (SeLFIE, also known as benign familial infantile epilepsy or BFIE) with normal cognition and medically treatable seizures; neurodevelopmental delays with generalized epilepsy (NDDwGE); and neurodevelopmental disorder without epilepsy (NDDwoE) with mild-to-moderate intellectual disability (which can be severe in ˜10% of affected individuals). Hypotonia and movement disorders including dystonia, ataxia, and choreoathetosis are common in some phenotypes. Sudden unexpected death in epilepsy (SUDEP) has also been reported in some affected individuals.

SCN8A is comprised of 28 exons with alternative splicing of both exon 5 (isoform 5A or 5N) and 18 (isoform 18A or 18N) (Plummer, N. W. et al., 1997, J Biol Chem, 272(38):24008-15; Plummer, N. W. et al., 1998, Genomics, 54(2):287-96). The 5N isoform of the SCN8A exon 5 is expressed during early development through early childhood, while the 5A isoform is expressed postnatally and during adulthood. Thirty-five patients with mutations in SCN8A exon 5 have been reported. Modulating gene product splicing from one exon 5 isoform to the alternative exon 5 isoform could allow for recovered function of SCN8A and reduce seizure occurrence in patients with mutations in exon 5.

In addition, findings have shown that Nav1.6 encoded by the 5A exon of SCN8A is less active than Nav1.6 encoded by the 5N exon of SCN8A (Vanoye, C. G. et al., 2022, Biophysical Journal, 121(3):94a-95a). Therefore, using the ASOs as described herein to increase the inclusion of exon 5A for encoding sodium ion channel Nav1.6 could, in turn, reduce the overall activity of Nav1.6 in a specific way. This would be a beneficial approach to use for treatment of the approximately 500 patients having with gain of function mutations or genetic alterations in SCN8A by specifically decreasing the Nav1.6 sodium channel activity in those patients with SCN8A gain of function mutations or alterations.

For epilepsy treatment, one or more of the ASO compounds as described herein can serve as a therapeutic agent that provides disease-modifying correction when administered to patients having one or more mutations in the 5N exon (or 5A exon) of SCN8A associated with neurological, neurodevelopmental, or neurodegenerative disorders, diseases, pathologies, or conditions.

### Cancers

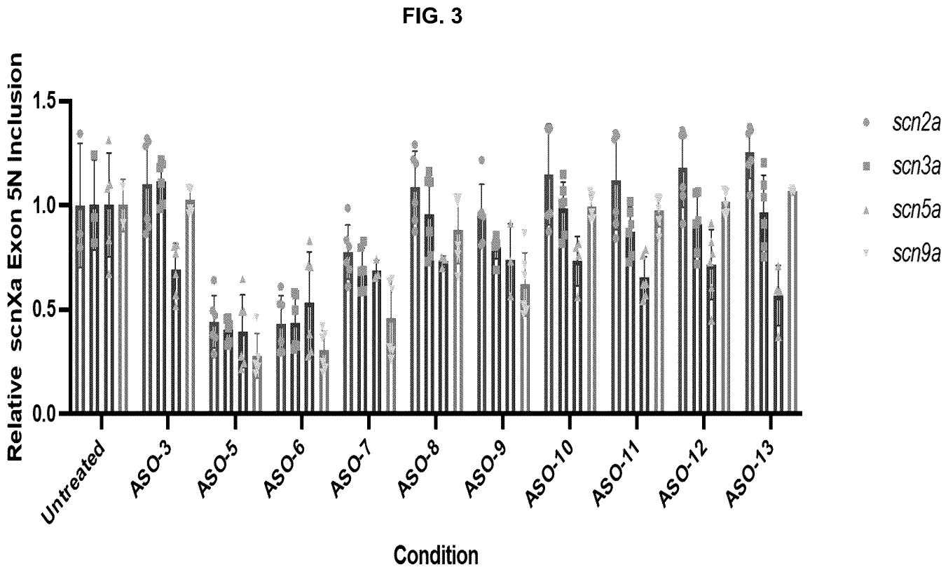

Splice variants exist across sodium ion channels. The SCN1A, SCN2A, SCN3A, SCN5A, and SCN9A genes have been reported to have an alternatively spliced exon 5 (5A or 5N), similar to that described for the SCN8A gene. (Liang, L. et al. 2021, Genome Medicine, 13, No. 135 (doi.org/10.1186/s13073-021-00949-0). Of interest, the neonatal isoform of exon 5 (5N) of the SCN5A gene has been shown to be expressed in breast cancer, colon cancer, astrocytomas, and neuroblastoma, relative to healthy tissue (Djamgoz, M. B. A. et al., 2019, Cancers (Basel), 11(11):1675). In addition, neonatal Nav1.5 (encoded by SCN5A) expression potentiates and increases the invasive potential of human breast tumor cells (Brackenbury, W. J. et al., 2007, Breast Cancer Res Treat, 101(2):149-60), and the inhibition of Nav1.5 reduces metastasis in vivo mouse models (Yang, M. et al., 2012, Breast Cancer Res Treat, 134(2):603-15; Nelson, M. et al., 2015, Oncotarget, 6(32):32914-29). Therefore, being able to downregulate the activity of Nav1.5 using a splice-switching ASO has advantageous and beneficial use in reducing invasion and metastasis. In an embodiment, the antisense oligonucleotides described herein may be used for targeting the genes (e.g., the exon 5 isoforms 5N/5A) that encode sodium ion channels associated with diseases and disorders such as cancer (e.g., SCN5A, SCN8A) for use as anti-cancer treatments and therapies. (Lopez-Charcas, O. et al., 2021, iScience, 24(4):102270). In an embodiment, ASO-11 of sequence accctgaaagtgcgta and ASO-13 of sequence ccctcagtaccctgaa, as set forth in Table 1, specifically target, bind, and modulate alternative splicing of SCN5A RNA, thereby specifically decreasing the inclusion of exon 5N of the SCN5A gene (FIG. 3).

### Therapeutic Approaches

Differential splicing patterns can be effective therapeutic targets in humans, for example through antisense oligonucleotides (ASOs), e.g., intrathecal ASOs. ASOs have proved useful for modifying gene-wide expression in rodent models of some voltage-gated sodium channel disorders (Liang, L. et al, 2021, Genome Medicine, 13, No. 135 (doi.org/10.1186/s13073-021-00949-0). Manipulating the expression of specific exons in sodium ion channel-encoding genes may represent a therapeutic strategy for treating neurological, neurodevelopmental, or neurodegenerative disorders, diseases, or pathologies, as well as cancers, or may represent a complementary therapeutic strategy used in conjunction with other drugs, reagents, and regimens for treating neurological, neurodevelopmental, or neurodegenerative disorders, diseases, or pathologies, as well as cancers.

For individuals with disorder-associated genetic variants within the 30 amino acids encoded by either the 5A or 5N isoform of exon 5, expressing the other copy of 5A/5N could skip the variant. This approach could benefit individuals with both loss-of-function variants (e.g., protein-truncating variants, missense variants, splice site variants) and severe gain-of-function (missense) variants. By way of example, at least eight cases of epileptic encephalopathy have been identified with variants in 5A of SCN2A or SCN8A (Liang, L. et al., 2021, Ibid.). Epilepsy resulting from many of these variants is poorly managed with antiepileptic drugs, which either block sodium channels with limited isoform specificity or target other mechanisms (e.g., other ion channels). The alteration of splicing to induce the exclusion of the exon 5N isoform using the ASOs described herein can be useful as a monotherapy or can also be combined, for a possible increased effect, with decreasing or reducing overall channel expression or expression of the normal alleles. In addition, the alteration of splicing to increase inclusion of exon 5A in Nav1.6 can reduce the overall activity of Nav1.6 in a specific way as a therapeutic treatment for patients with mutations or genetic alterations in SCN8A, such as gain-of-function mutations or in missense variants. (See, e.g., Johannesen, K. M. et al., 2022, Brain, 145(9):2991-3009). By way of a related example, haploinsufficiency of sodium ion channel encoding genes (e.g., SCN2), is associated with autism spectrum disorder and intellectual disability in a subset of patients. Because some children with SCN42A haploinsufficiency experience seizures, lowering the SCN22 expression levels below 50% could increase the prevalence of such seizure conditions. Therefore, for this subset of patients, splice-altering ASOs may provide a wider therapeutic window than ASOs that reduce gene expression. It will be appreciated that the success of such a therapy will depend upon the proportion of transcripts that express the alternate 5th exon and the ability of this exon to functionally replace the original 5th exon. In addition, mutations in disease (epilepsy)-associated SCN2A variants involving exon 5N and 5A and alternatively spliced forms of Nav1.2 were found to have gain-of-function and loss-of-function properties. (Thompson, C. H. et al., 2023, bioRxiv, pp. 1-34; doi.org/10.1101/2023.02.23.529757).

In SCN8A, gain-of-function variants lead to epileptic encephalopathy. At least 500 individuals are known to be afflicted with SCN84 gain-of-function mutations or genetic alterations. The antisense oligonucleotides described herein can serve as disease-modifying agents for specifically reducing or decreasing the activity of the encoded Nav1.6 channel in patients having gain-of-function SCN8A mutations to improve symptoms and treat the disorder. Interventions that promote switching from exon 5N to 5A (or that promote exon skipping of 18A or exon switching from 18A to 18N) would prevent translation of functional Nav1.6 channels, serving to treat disease and improve symptoms. All interventions aimed at reducing SCN8A levels would require careful dosing since SCN8A haploinsufficiency is associated with intellectual disability.

In addition, by way of a related example, three recently characterized epileptic encephalopathy-associated variants in SCN2A, namely, T236S, E999K, and S1336Y, were found to exhibit more pronounced alterations in their electrophysiological properties in 5N Nav1.2 isoforms compared to 5A isoforms (Thompson, C. H. et al., 2020, J Gen Physiol, 152(3):1-16). For individuals with these types of variants, or equivalent variants, driving expression toward the 5A isoform, e.g., by reducing levels of the 5N isoform, could provide some symptomatic improvement, especially during infancy.

Methods of Treating Diseases and Disorders Associated with Mutations or Genetic Alterations in Exon 5 of the SCN8A Gene

In the aspects and embodiments herein, methods of treating a subject having or suspected of having sodium ion channel disease, disorder, pathology, or condition, or a disease, disorder, pathology, or condition associated with a sodium ion channel encoded by exon 5N or exon 5A of SCN8A, and/or symptoms thereof, are provided by administering one or more of the antisense oligonucleotides as described herein. In some embodiments, methods are provided for treating or preventing the disease, disorder, pathology, or condition, or symptoms thereof, that comprise administering a therapeutically effective amount of a pharmaceutical composition comprising the antisense oligonucleotides disclosed herein to a subject or individual (e.g., a mammal such as a human). As will be appreciated by those having skill in the art, the pharmaceutical composition comprises a pharmaceutically acceptable, or physiologically acceptable excipient, carrier, or diluent.

Administering the therapeutically effective amount of an antisense oligonucleotide as described herein comprises administering an amount sufficient to treat the disease, disorder, pathology, or condition, or a symptom thereof, under conditions such that the disease, disorder, pathology, or condition is treated. In embodiments, treatment involves ameliorating, reducing, abating, diminishing, alleviating, or eliminating the disease, disorder, pathology, or condition, or a symptom thereof. In an embodiment, the disease or disorder is a neurological, neurodevelopmental, or neurodegenerative disorder, disease, or pathology, or symptom thereof. Nonlimiting examples of such neurological, neurodevelopmental, or neurodegenerative disorders, diseases, or pathologies include mild to severe intellectual disabilities, autism spectrum disorders, movement disorders, developmental and epileptic encephalopathy (DEE), epilepsy, epileptic seizures, and Sudden Unexpected Death in Epilepsy (SUDEP). In an embodiment, the disease or disorder is a cancer, or symptom thereof. Nonlimiting examples of cancers include breast cancer, colon cancer, brain cancers, astrocytomas, gliomas, and neuroblastoma. Other cancers are encompassed, such as ovarian cancer, cervical cancer, lung cancer, kidney cancer, prostate cancer, pancreatic cancer, liver cancer, rectal cancer, esophageal cancer, gastrointestinal cancer, testicular cancer, sarcomas, leukemias (B and T cell leukemias), lymphomas, cancers of the immune system, and the like. In an embodiment, the cancer is one in which the neonatal isoform (5N) of exon 5 of SCN5A is expressed leading to neonatal Nav1.5 expression.

Administration of antisense oligonucleotides to a subject having or suspected of having a disease or disorder associated with a 5A/5N exon 5 isoform of a sodium ion channel-encoding gene can be targeted or systemic. Generally, the antisense oligonucleotide will be administered in a pharmaceutical composition as described above.

In some embodiments, a composition comprising the antisense oligonucleotides described herein is conveniently presented in unit dosage form and is prepared by any method well known in the art. The dosage of the administered antisense oligonucleotide depends on a number of factors, including the size and health of the individual patient. For any particular subject, the specific dosage regimes should be adjusted over time according to the individual need and the professional judgment of the person administering or supervising the administration of the compositions. The amount of active ingredient that can be combined with a carrier material to produce a single dosage form will vary depending upon not only the host being treated but also the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, from about 5 percent to about 70 percent, or from about 10 percent to about 30 percent.

Additional suitable carriers and their formulations are described, for example, in the most recent edition of Remington's Pharmaceutical Sciences by E. W. Martin. The amount of the therapeutic agent to be administered varies depending upon the manner and mode of administration, the age and disease status (e.g., the extent of hearing loss present prior to treatment).

Compositions are administered at a dosage that controls the clinical or physiological symptoms of the disease or condition, as may in some cases be determined by a diagnostic method known to one skilled in the art, or using any assay that measures the biological activity of an antisense oligonucleotide or composition comprising an antisense oligonucleotide, or a combination thereof.

Therapeutic compounds and therapeutic combinations are administered in an effective amount. In certain embodiments, compositions, such as those described herein, are administered at dosage levels of about 0.0001 to 1.0 g once per day (or multiple doses per day in divided doses). In certain embodiments, an antisense oligonucleotide or composition as described herein is administered at a dosage between 0.1 mg/day and 100 mg/day and the upper end of the range is any amount between 1 mg/day and 1000 mg/day (e.g., 5 mg/day and 100 mg/day, 150 mg/day and 500 mg/day). In other embodiments, a compound or composition as herein is administered at a dosage range in which the low end of the range is any amount between 0.1 mg/kg/day and 50 mg/kg/day and the upper end of the range is any amount between 1 mg/kg/day and 100 mg/kg/day (e.g., 0.5 mg/kg/day and 2 mg/kg/day, 5 mg/kg/day and 20 mg/kg/day). In some embodiments, a composition as described herein is administered at a dosage of about 10 mg/dose, about 20 mg/dose, about 30 mg/dose, about 40 mg/dose, about 50 mg/dose, about 60 mg/dose, about 70 mg/dose, about 80 mg/dose, about 90 mg/dose, about 100 mg/dose, about 110 mg/dose, about 120 mg/dose, about 130 mg/dose, about 140 mg/dose, about 150 mg/dose, about 160 mg/dose, about 170 mg/dose, about 180 mg/dose, about 190 mg/dose, or even about 200 mg/dose. In some embodiments, a composition as described herein is administered at a dosage of between 1 and 10 mg/dose. For example, in some embodiments, a composition as described herein is administered at a dosage of about 1, 2, 3, 4, 5, 6, 7, 8, 9, or 10 mg/dose.

In some embodiments, administration may not be daily, but rather once every 2, 4, 6, 8, or 10 weeks. For example, in administering antisense oligonucleotides to children, the dosage regimen may be between 1 and 50 mg every two weeks for 8 to 10 weeks followed by 1 to 100 mg every four weeks for 3 to 4 weeks, followed by every 8 weeks for 16 to 48 weeks. The dosing interval can be adjusted according to the needs of individual patients.

### Antisense Oligonucleotide-Mediated Exclusion of Voltage Gated Sodium Ion Channel-Encoding Gene Isoforms

Antisense oligonucleotides are provided that impact the splicing of SCN8A exon 5 and induce the exclusion of exon 5N in the Nav1.6 sodium ion channel polypeptide. In an embodiment, an antisense oligonucleotide as described herein comprises 2′-modified oligonucleotides where some or all internucleotide linkages are modified to phosphorothioates or phosphodiester (PO). In some embodiments, the presence of methylphosphonate modifications increases the affinity of the oligonucleotide for its target RNA and thus reduces the IC₅₀. This modification also increases the nuclease resistance of the modified oligonucleotide. As will be appreciated by one skilled in the art, the reagents, agents, and methods as described herein may be used in conjunction with any additional technologies or therapies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′, or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred ASOs as described herein include oligonucleotides containing modified backbones or non-natural internucleoside linkages. Nucleobase oligomers having modified backbones include those that retain a phosphorus atom in the backbone and those that do not have a phosphorus atom in the backbone. In some embodiments, modified oligonucleotides that do not have a phosphorus atom in their internucleoside backbone are also considered to be nucleobase oligomers.

ASOs, i.e., antisense oligonucleotides, (also called nucleobase oligomers), that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

ASOs having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. One such nucleobase oligomer, is referred to as a peptide nucleic acid (PNA). In PNA compounds, the sugar-backbone of an oligonucleotide is replaced with an amide containing backbone, such as an amino-ethylglycine backbone. The nucleobases are retained and are bound directly or indirectly to aza nitrogen atoms of the amide portion of the backbone. Methods for making and using these antisense oligonucleotides are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is incorporated by reference herein. Other information concerning PNA compounds may be found in Nielsen et al., 1991, Science, 254: 1497-1500.

In particular embodiments, the ASOs have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O—, S—, or N-alkyl; O—, S—, or N-alkenyl; O-, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly useful are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other nucleobase oligomers include one of the following at the 2′ position: C1 to C10 lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Exemplary 2′ modifications are 2′-O-methyl and 2′-methoxyethoxy (2′-O—CH₂CH₂OCH₃, also known as 2′-O-methoxyethyl (2′-MOE). Other desirable modifications include 2′-dimethylaminoethoxyethoxy and 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂), 2′-fluoro (2′-F), and 2′-acetamide. Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. In some embodiments, a nucleobase oligomer comprises a locked nucleic acid (LNA). Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G) and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-amino adenine, 6-methyl, and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo (e.g., 5-bromo), 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional nucleobases include those disclosed in U.S. Pat. No. 3,687,808, those disclosed in The Concise Encyclopedia of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Certain nucleobases are particularly useful for increasing the binding affinity of an antisense oligonucleotide as described herein. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6° C.-1.2° C. (Sanghvi, Y. S., Crooke, S. T. and Lebleu, B., eds., Antisense Research and Applications, CRC Press, Boca Raton, 1993, pp. 276-78) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,681,941; and 5,750,692, each of which is herein incorporated by reference.

Another modification of an ASO as described herein involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-60, 1994), a thioether, e.g., hexyl-S-tritylthiol (Manoharan et al., Ann. N. Y. Acad. Sci., 660:306-09, 1992; Manoharan et al., Bioorg. Med. Chem. Let., 3:2765-70, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-18, 1991; Kabanov et al., FEBS Lett., 259:327-30, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-54, 1995; Shea et al., Nucl. Acids Res., 18:3777-83, 1990), a polyamine or a polyethylene glycol chain (Manoharan et al., Nucleosides & Nucleotides, 14:969-73, 1995), or adamantane acetic acid (Manoharan et al., Tetrahedron Lett., 36:3651-54, 1995), a palmityl moiety (Mishra et al., Biochim. Biophys. Acta, 1264:229-37, 1995), or an octadecylamine or hexylamino-carbonyl-oxycholesterol moiety (Crooke et al., J. Pharmacol. Exp. Ther., 277:923-37, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241; 5,391,723; 5,414,077; 5,416,203; 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

Also encompassed herein are nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with the embodiments described herein may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

### Compositions and Formulations Containing the ASOs

The ASOs of the described aspects and embodiments herein may also be admixed, encapsulated, conjugated, or otherwise associated with other molecules, molecule structures or mixtures of compounds, as for example, liposomes, receptor targeted molecules, oral, rectal, topical, or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense oligonucleotides (nucleobase oligomers) of the described embodiments encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the described compounds, pharmaceutically acceptable salts of such prodrugs, and other bioequivalents.

The term “pharmaceutically acceptable salts” refers to salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., Berge et al., J. Pharma Sci., 66:1-19, 1977). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes described herein. As used herein, a “pharmaceutical addition salt” includes a pharmaceutically acceptable salt of an acid form of one of the components of the compositions of the disclosed embodiments. These include organic or inorganic acid salts of the amines. In embodiments, the acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides and other nucleobase oligomers, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

Pharmaceutical compositions and formulations that include the antisense oligonucleotides (nucleobase oligomers) are encompassed by the aspects and embodiments described herein. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral, or parenteral. Parenteral administration includes intravenous, intraarterial, subcutaneous, intraperitoneal, or intramuscular injection or infusion; or intracranial, e.g., intrathecal or intraventricular, administration. In a particular embodiment, intrathecal administration is employed.

### Screening for Agents to Recover Wild-Type Splicing of a Gene

In one aspect, methods of screening for antisense oligonucleotides that decrease exon 5N or increase exon 5A of SCN8A encoding the sodium ion channel Nav1.6 in cells or tissues are provided. Thus, in various embodiments, the method screens for an antisense oligonucleotide that impacts the splicing of SCN8A exon 5, inducing the exclusion of exon 5N and decrease in its inclusion, in cells having a mutated sodium ion channel polypeptide (e.g., Nav1.6)-encoding gene, such as SCN8A.

Numerous methods are also available for generating random or directed synthesis (e.g., semi-synthesis or total synthesis) of oligonucleotides. The screening method presented herein may be amendable to a high throughput format.

In some embodiments, antisense oligonucleotides are synthesized that are complementary to an exon 5N nucleic acid sequence in SCN8A that contains a mutation that is associated with a disease, disorder, pathology, or condition as described herein. The antisense oligonucleotides can be transfected into cells (e.g., a neuronal cell or cell line) comprising an SCN8A exon 5N isoform and/or mutation or variant in the SCN8A gene. Transcripts can be analyzed to determine the presence and quantity of the exon 5N isoform that is produced. In other embodiments, antisense oligonucleotides are synthesized that are complementary to an exon 5A nucleic acid sequence in SCN8A that contains a mutation that is associated with a disease, disorder, pathology, or condition as described herein. The antisense oligonucleotides can be transfected into cells (e.g., a neuronal cell or cell line) comprising an SCN8A exon 5A isoform and/or mutation or variant in the SCN8A gene. Transcripts can be analyzed to determine the presence and quantity of the exon 5A isoform that is produced. Methods of detecting and characterizing mRNA transcripts are known in the art, such as oligonucleotide microarray assays, quantitative RT-PCR, Northern analysis, and multiplex bead-based assays. In some embodiments, RT-PCR can be used to amplify the transcripts, and the resulting amplification products can be visualized on a gel or by any other means known in the art.

An antisense oligonucleotide identified as being able to induce the exclusion or decrease the activity of the exon 5N or the exon 5A isoform of SCN8A (i.e., a lead antisense oligonucleotide) can be optimized to increase the percentage of exon 5N or of exon 5A activity and/or increase efficiency of splicing to obtain the exon 5N isoform or the exon 5A isoform, or an increased amount thereof in a cell. For example, in some embodiments, additional antisense oligonucleotides (“optimized antisense oligonucleotides”) that have substantial sequence identity to the lead antisense oligonucleotide are generated. In some embodiments, the optimized antisense nucleotides include modified nucleobases or backbones that enhance binding to RNA transcripts. In other embodiments, the optimized antisense oligonucleotides differ in length from the lead antisense oligonucleotide.

In some embodiments, an antisense oligonucleotide's ability to increase the percentage of 5N isoform activity and/or to increase efficiency of splicing to obtain the 5N isoform or enhanced amount of the isoform can be assessed using the assay as described herein in Example 2 to detect the ratio of the 5N:5A isoforms in the cell. In some embodiments, an antisense oligonucleotide's ability to increase the percentage of 5A isoform activity and/or to increase efficiency of splicing to obtain the 5A isoform or enhanced amount of the isoform can be assessed using the assay as described herein in Example 2 to detect the ratio of the 5A:5N isoforms in the cell.

### Downregulation of the Activity of a Voltage Gated Sodium Ion Channel Protein Using a Splice-Switching ASO

Mutations that are associated with the transcription of the exon 5N isoform of the SCN8A gene encoding the Nav1.6 voltage-gated sodium ion channel polypeptide have been identified. Provided herein are methods of decreasing the activity of, or inducing or increasing the exclusion of exon 5N, or decreasing the activity of the Nav1.6 sodium ion channel in a cell, such as a cell that has a mutation in SCN8A that results in a disease, disorder, pathology, or condition. Also provided are methods of decreasing the activity of, or inducing or increasing the exclusion of exon 5A, related to the activity of the Nav1.6 sodium ion channel in a cell. In an embodiment, the mutation in SCN8A is a gain-of-function mutation. In some embodiments, the methods comprise contacting a cell having a mutation in the SCN8A gene that is associated with a disease, disorder, pathology, or condition (e.g., a neurological, neurodevelopmental, or neurodegenerative disease, disorder, pathology, or condition) with one or more antisense oligonucleotides described herein. In some embodiments, the antisense oligonucleotide can be an antisense oligonucleotide in Table 1 or Table 2. In some embodiments, the antisense oligonucleotide has one or modification described herein. In an embodiment, the exclusion of SCN8A exon 5N expression results in an increase in expression of SCN8A exon 5A expression. In an embodiment, the exclusion of SCN8A exon 5A expression results in an increase in expression of SCN8A exon 5N expression. In embodiments, the average exon 5N inclusion or exon 5A inclusion is reduced by at least 2-fold, at least 3-fold, at least 5-fold, at least 6-fold, at least 7-fold, at least 8-fold, at least 9-fold, at least 10-fold, at least 15-fold, at least 20-fold, or by at least 25-fold or greater, including values therebetween, in a cell containing or subjected to contact with an antisense oligonucleotide as described herein (Tables 1 to 3) compared with an untreated cell. In embodiments, the antisense oligonucleotides are ASOs 3 and 5-13 of Table 1. In a particular embodiment, exon 5N inclusion drops from 83% to an average of 16% through the use of an ASO as described herein, e.g., ASO 3, or any of ASO 5-13 of Table 1. In embodiments, the antisense oligonucleotides are ASOs 24 and 27 of Table 2 and Table 3.

In some embodiments, contacting the cell with an antisense oligonucleotide as described herein can result in about 10%, about 20%, about 30%, about 40%, about 50%, about 60%, about 70%, about 80%, about 90%, or about 100% of all exon 5 transcripts being of the exon 5A isoform or the exon 5N isoform compared with an untreated cell. In some embodiments, the contacted cell is a neuronal cell (neuron), a glial cell, brain cell, a cortical neuron, a primary cortical neuron, a cell of the central nervous system (CNS), a gliobastoma cell, an astrocytoma cell, a cell obtained or isolated from a subject with a disease, disorder, pathology, or condition associated with a mutation in SCN8A, and the like.

In some embodiments, the cell is a cell whose function is impaired by a mutant or variant form of SCN8A. In some embodiments, the contacting is in vitro or ex vivo. In some embodiments, the contacting is in vivo. In some embodiments, the cell comprises a gain-of-function mutation in the SCN8A gene. In some embodiments, the antisense oligonucleotide can be designed to hybridize with a splice acceptor or a splice donor or a splicing regulatory element.

### Kits and Articles of Manufacture

Provided also are kits and articles of manufacture for use in the treatment or prevention of a disease, disorder, pathology, or condition, or symptoms thereof, associated with a variant or mutated sodium ion channel-encoding gene, for example, without limitation, SCN8A or SCN5A. In an embodiment, the SCN8A variant or mutant form involves alternative splicing of exon 5 or exon 5 isoform 5A or 5N of the SCN8A gene. In an embodiment, the SCN5A variant or mutant form involves alternative splicing of exon 5 or exon 5 isoform 5A or 5N of the SCN5A gene. In some cases, the SCN8A gene or exon 5 isoform is aberrantly, abnormally, or improperly expressed. In some cases, the exon 5 isoform is a variant or is a mutated form of SCN8A. In some cases, inhibition, reduction, or downregulation of activity of a sodium ion channel, e.g., Nav1.6, encoded by exon 5N of the SCN8A gene may serve as treatment for a neuronal, neurodegenerative, neurodevelopmental, or neuropathological disease or disorder associated with expression, or overexpression, of the sodium ion channel encoded by exon 5N. In some cases, inhibition, reduction, or downregulation of activity of a sodium ion channel, e.g., Nav1.5, encoded by exon 5N of the SCN5A gene may serve as treatment for cancers, e.g., breast cancer, metastatic breast cancer, associated with expression, or overexpression, of the sodium ion channel encoded by exon 5N of SCN5A. The ability to downregulate the activity of Nav1.5 using a splice-switching ASO as described herein has advantageous and beneficial use in reducing invasion and metastasis of cancer and tumor cells.

In one embodiment, the kit or article of manufacture includes a pharmaceutical pack, or a set or panel, comprising an effective amount of one or more antisense oligonucleotides (ASOs) as described herein. In some embodiments, the compositions are in unit dosage form. In some embodiments, the kit or article of manufacture comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions or combinations thereof are provided together with instructions for administering them to a subject having or at risk of developing a disease or disorder as described above. The instructions will generally include information about the use of the compounds for the treatment or prevention of the disease or disorder. In other embodiments, the instructions include at least one of the following: description of the compound or combination of compounds; dosage schedule and administration for treatment of the disease or disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the aspects and embodiments described in the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides as described herein, and, as such, may be considered in making and practicing the described aspects and embodiments. Particularly useful techniques for particular embodiments will be discussed in the sections that follow.

The following examples are put forth so as to provide those of ordinary skill in the art with a complete disclosure and description of how to make and use the compounds, reagents, assay, screening, and therapeutic methods as described, and are not intended to limit the scope of the disclosed and described aspects and embodiments. All publications cited herein are incorporated by reference for the purposes or subject matter referenced herein.

### EXAMPLES

### Example 1: Antisense Oligonucleotide (ASO) Design

Antisense oligonucleotides (ASOs) were designed to bind to SCN8A exon 5N and were generated using PFRED software as described in Sciabola, S. et al., 2021, PFRED: A computational platform for siRNA and antisense oligonucleotides design, PLoS One, 16(1): p. e0238753. Eighteen ASOs (ASO-1 to ASO-18) generated and used for screening were 16-mers, with 2′-O-methoxyethyl (2′-MOE) and phosphorothioate (PS) backbones throughout the entirety of the sequence. In addition, ASOs, e.g., ASO-24 and ASO-27, were designed and generated to bind to SCN8A exon 5A. The sequences and backbone sequences of the exon 5A-binding ASOs, ASO-24 and ASO-27, are presented in Table 3 in Example 5 hereinbelow. The sequences of the oligonucleotides are provided in Table 1, reproduced below. Integrated DNA Technologies (Coralville, IA), a vendor service, constructed the ASOs based on the sequences provided to them

| Name | Sequence (5′ to 3′) |
|---|---|
| scn8a_ASO-1 | cttgtaatggtatcac |
| scn8a_ASO-2 | aatggtaccactgggc |
| scn8a_ASO-3 | ctgcagaatcaaacca |
| scn8a_ASO-4 | gcctaggtttacaaac |
| scn8a_ASO-5 | gcgtagagctgaaaca |
| scn8a_ASO-6 | gtgcgtagagctgaaa |
| scn8a_ASO-7 | aagtgcgtagagctga |
| scn8a_ASO-8 | gaaagtgcgtagagct |
| scn8a_ASO-9 | ctgaaagtgcgtagag |
| scn8a_ASO-10 | ccctgaaagtgcgtag |
| scn8a_ASO-11 | accctgaaagtgcgta |
| scn8a_ASO-12 | agtaccctgaaagtgc |
| scn8a_ASO-13 | ccctcagtaccctgaa |
| scn8a_ASO-14 | gggattaccgaaatag |
| scn8a_ASO-15 | ctgggattaccgaaat |
| scn8a_ASO-16 | acccaacacctgacac |
| scn8a_ASO-17 | ctaacccaacacctga |
| scn8a_ASO-18 | gccctaacccaacacc |

### Example 2: Assay to Determine the Ratio of Exons 5A:5N of SCN8A

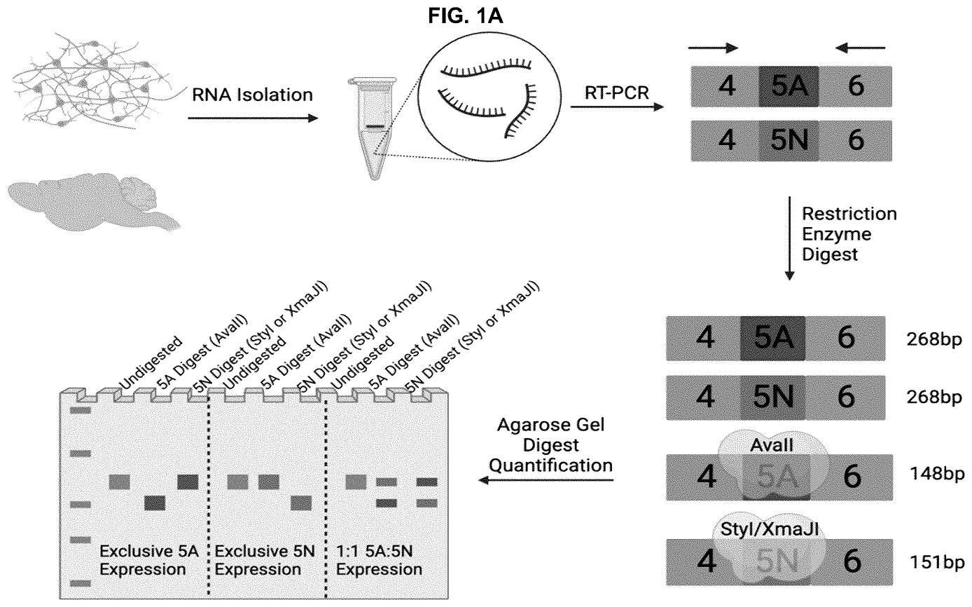

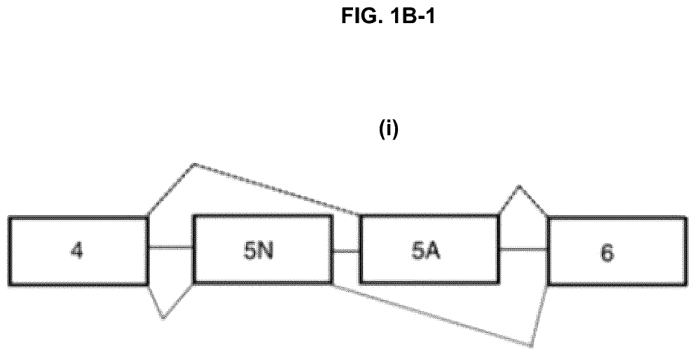

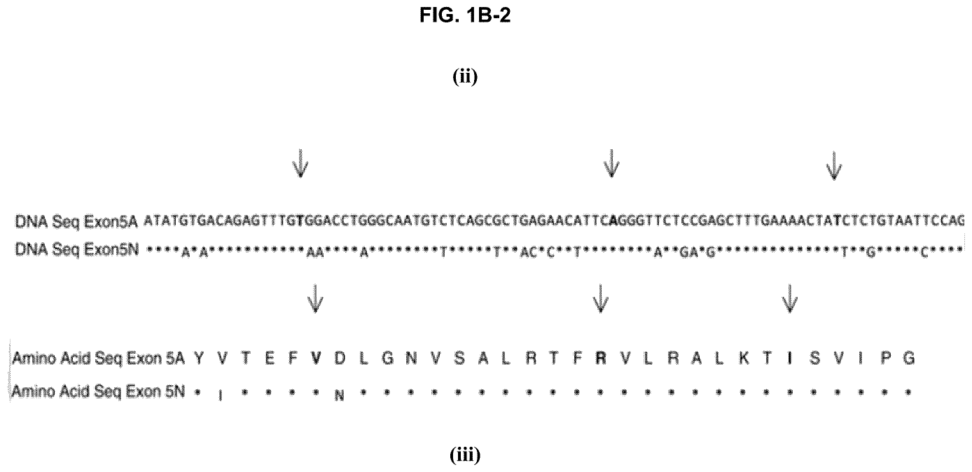

Once designed, the oligonucleotides described in Example 1 were evaluated for activity. To study the alternative splicing of SCN8A exon 5, a restriction digest assay was developed that allowed for the exclusive digestion of isoform 5A or isoform 5N of exon 5 of SCN8A by determining the ratio of the 5A:5N isoforms in cells of a cultured cell line, in mouse brain tissue, or in cultured primary neurons (FIG. 1A). FIGS. 1B-1, 1B-2, and 1B-3 illustrate structural and sequence information of Exon 5, the 5N and 5A isoforms of the SCN8A gene, and its encoded transmembrane sodium ion channel polypeptide. (Epilepsy Genetics Initiative, 2018, Genet Med, 20(2):275-281).

The cultured cell line used was ND7/23, a mouse neuroblastoma/rat neuron hybrid, available from Millipore/Sigma (cell line 92090903) as described in Example 3 below. As depicted in FIG. 1A, total RNA was isolated from cultured cells or brain tissue using the Zymo Quick-RNA kit (ZYMO Research, Irvine, CA), which yields highly concentrated, DNA-free RNA for performing RNA-based methods, including RT-PCR, etc. One step RT-PCR with primers specific to SCN8A exons 4 and 6 was performed on the isolated RNA, resulting in a PCR product of 268 bp. Following the RT-PCR reaction, the PCR product was digested using the restriction enzyme AvaII, which specifically targeted and cleaved the SCN8A exon 5A isoform, or using restriction enzyme StyI, which specifically targeted and cleaved the SCN8A exon 5N isoform. Enzymatic digestions of the PCR product with the specific restriction enzymes resulted in digested band sizes near 210 bp following electrophoresis on an agarose gel. The digested and undigested PCR products were subjected to agarose gel electrophoresis for quantification. A 3% agarose gel was used. Following a gel run of 2+ hours, the bands resulting from the digests were easily distinguishable and were quantified using ImageJ gel analysis. The percent digestion was determined based on the area of the band on the agarose gel. Percent digested was determined by dividing the digested or undigested band area by the total band area, which is the sum of the digested and undigested band areas. For example, if the StyI enzyme (specific for the 5N isoform) was used, and there was a digested band with an area of 75 and an undigested band with an area of 25, then it was calculated that the sample expressed 75% of the 5N isoform and 25% of the 5A isoform.

The gel shown in FIG. 1A demonstrates different digestion results obtained. In the left portion of the gel, the first three lanes represent the results found if the cells/tissue sample exclusively expressed the exon 5A isoform. Thus, when AvaII (5A targeting) digestion occurs, the entire PCR product is digested by the enzyme, but when StyI (5N targeting) is used, no digestion occurs. The three lanes in the middle portion of the gel represent the results found if the cells/tissue exclusively expressed the 5N isoform, where digestion occurs only when the 5N targeting enzyme StyI is used. The last three lanes in the rightmost portion of the gel depict the results found if the cells/tissue expressed a 1:1 ratio of the 5A:5N exon 5 isoforms. In the last three lanes, equal band areas represent digested and undigested when both the 5A and 5N isoform targeting enzymes are used.

### Example 3: Antisense Oligonucleotide (ASO) Modification of SCN8A Exon 5 Isoforms in ASO-Treated Cells

To assess the activities of the ASOs created and generated as described herein, ND7/23 cells (Millepore/Sigma 92090903) were plated at 30-50% confluency in a 12-well tissue culture plate in 1×DMEM medium supplemented with 10% FBS and 1× Penicillin-Streptomycin-Glutamine (PSG). Cells were then treated with 500 nM of an ASO as described herein and transfected using lipofectamine (Invitrogen L3000-008) in triplicate. Cells treated with an ASO were incubated for 24 hours. Following the 24-hour incubation, RNA was isolated from the cells using the Zymo RNA Mini-Prep Kit (Fisher Scientific 50-444-597). The RNA was then converted into cDNA using a ThermoFisher High-Capacity cDNA Reverse Transcription Kit (ThermoFisher 4368814). The cDNA was subjected to polymerase chain reaction (PCR) using a Phusion Green High-Fidelity DNA Polymerase Kit (ThermoFisher F534S), 1000 ng cDNA and SCN8A primers. The SCN8A forward primer was: 5′-tggaactggttagatttcagtgt-3′; the SCN8A reverse primer was: 5′-caacacacttgtttcgaaggtt-3′. After completion of the PCR, samples were treated with the restriction enzymes AvaII (ThermoFisher ER0311) or StyI (ThermoFisher ER0411) and were incubated at 37° C. for 1 hour. The digested samples were then electrophoresed on a 3% agarose gel for 2.5 hours. The agarose gel results were analyzed using ImageJ for agarose band areas of the 5A or 5N isoforms.

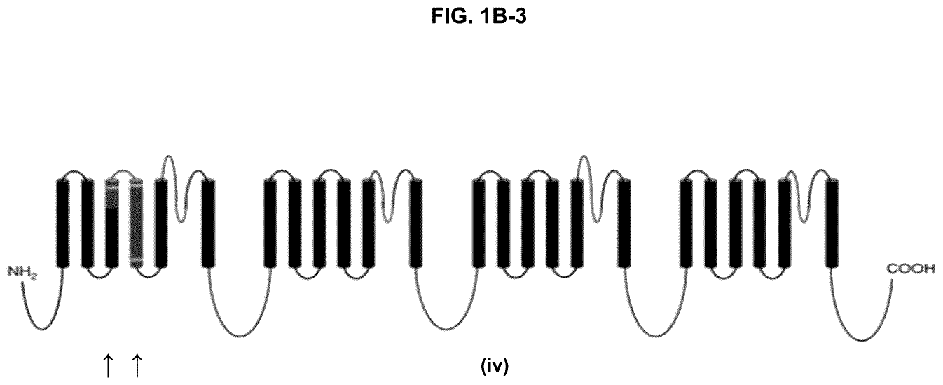

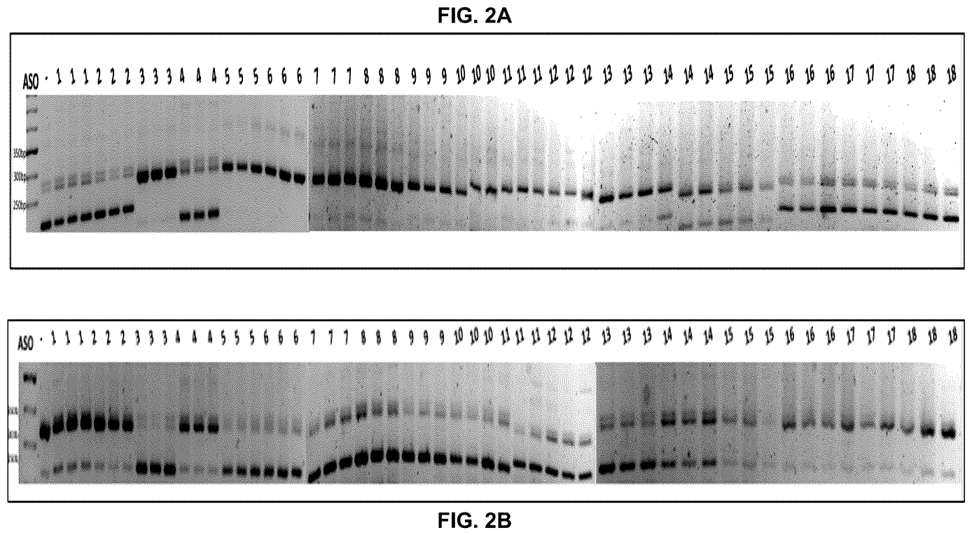

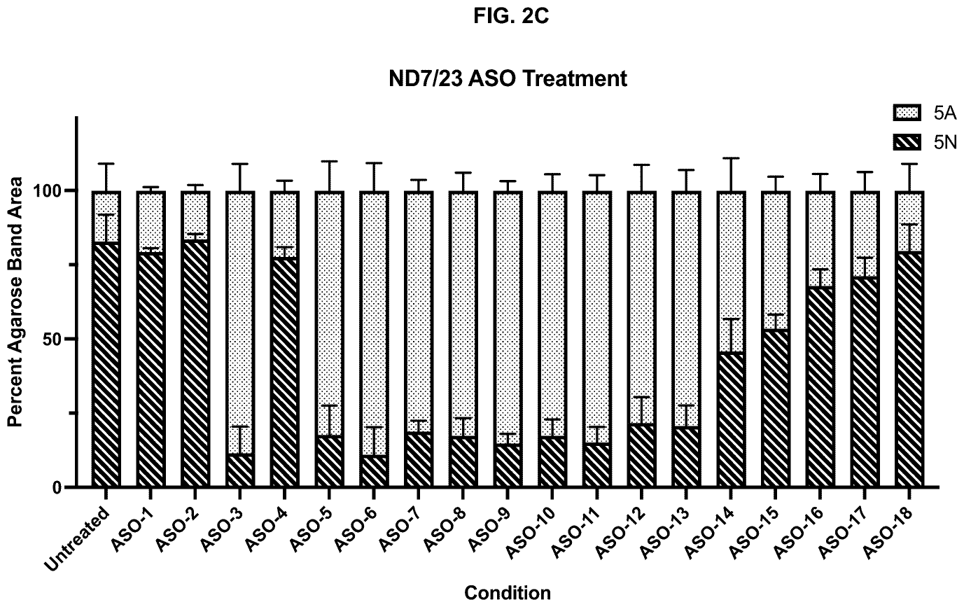

As shown in FIGS. 2A-C, untreated ND7/23 cells expressed the SCN8A 5N isoform at a level of 83%. Digestion of untreated ND7/23 cells was high using the restriction enzyme StyI, a 5N targeting restriction enzyme, resulting in a high intensity band of lower molecular weight (FIG. 2A). With the addition of an antisense oligonucleotide, namely, ASO numbers 3 and 5-13 (Table 1), a reduction in the digestion of SCN8A exon 5 was detected using the StyI enzyme, concluding that the ASOs are inducing SCN8A exon 5N exclusion (FIG. 2A). Untreated ND7/23 cells digested with AvaII, a 5A targeting restriction enzyme have a low intensity of digested band due to the high 5N inclusion within the cells (FIG. 2B). The intensity of the digested bands increased with the treatment of the ND7/23 cells with ASOs numbers 3 and 5-13, because these ASOs induced exon 5N exclusion of the SCN8A gene (FIG. 2B). Thus, these ASOs achieved the exclusion of the SCN8A 5N isoform, resulting in an increase in SCN8A 5A expression. ASO numbers 3 and 5-13 resulted in an average 5N isoform expression of 16% (FIG. 2C).

The results of the experiments showed that that the addition of an ASO of numbers 3, and 5-13 (Table 1) to cells impacted the splicing of SCN8A exon 5 and induced the exclusion of the exon 5N isoform in the cells, resulting in an increase in expression of the SCN8A 5A isoform. The exon 5N inclusion decreased from 83% to an average of 16%. In an embodiment, one or more of the ASOs described herein may be added to a cell.

### Example 4: ASOs Specific for Alternative Splicing of Exon 5 Isoform 5N of the SCN8A Gene

Alternative splicing of exon 5, which generates isoforms 5N and 5A, occurs in multiple sodium ion channel genes, e.g., SCN2A, SCN3A, SCN5A, SCN8A and SCN9A (Raymond C. K. et al., 2004, J Biol Chem., 279(44):46234-41. doi: 10.1074/jbc.M406387200. Epub 2004 Aug. 9. PMID: 15302875). While expression of SCN2A and SCN3A is high in the nervous system, SCN9A expression is localized in the periphery, and SCN5A expression is primarily in the heart. Because the exon isoform 5N has a high sequence similarity among the different sodium ion channel genes (“SCNXA”), experiments were performed to determine the specificity of the ASOs described herein for alternative splicing of exon 5N of the SCN8A gene. To investigate the specificity of the ASOs herein for specifically targeting and binding exon 5N of SCN8A and specifically inducing a decrease in exon 5N expression in the SCN8A gene, the 5A/5N ratio assay as described in Example 2 was conducted using primers specific to the SCN2A, SCN3A, SCN5A or SCN9A gene polynucleotide sequences.

To quantify the relative 5N inclusion of SCNXA genes following the treatment of cells expressing the genes with ASOs as described herein, ND7/23 cells were treated with the ASOs as shown in FIG. 3 (x-axis, “Condition”) compared to untreated control cells to assess alternative splicing of exon 5N of the sodium ion channel genes SCN2A, SCN3A, SCN5A and SCN9A. ND7/23 cells were treated or untreated for 24 hours with the different ASOs (500 μM) via lipofectamine transfection. RNA was isolated from the cells and was converted to cDNA, followed by PCR using primers specific to the sodium ion channel genes of interest (i.e., SCN2A, SCN3A, SCN5A and SCN9A). The PCR products were digested with restriction enzymes StyI (5N specific) and AvaII (5A specific), and the resulting product was electrophoresed on a 3% agarose gel and quantified using the publicly available ImageJ software tool.

Based on these experiments, ASO-8 (i.e., gaaagtgcgtagagct), ASO-10 (i.e., ccctgaaagtgcgtag), and ASO-12 (i.e., agtaccctgaaagtgc) were found to induce no significant change in 5N inclusion of the SCNXA genes relative to the untreated controls (FIG. 3). ASO-5 (i.e., gcgtagagctgaaaca) and ASO-6 (i.e., gtgcgtagagctgaaa) were found to induce a significant decrease in 5N inclusion for SCN2A, SCN3A, SCN5A and SCN9A. ASO 7 (i.e., aagtgcgtagagctga) was found to induce a significant decrease in 5N inclusion of the SCN3A and SCN5A genes. ASO-11 (i.e., accctgaaagtgcgta) and ASO-13 (i.e., ccctcagtaccctgaa) were found to induce a significant decrease in 5N inclusion of the SCN5A gene. These results showed that ASOs were generated that are specific to SCN8A, in that they induced no changes in exon 5N inclusion of the SCN2A, SCN3A, SCN5A or SCN9A genes. The experiments confirmed that ASOs were designed that specifically target and bind to exon 5N of SCN8A, and do not target and bind exon 5N of other alternatively-splicing sodium channel-encoding genes and do not induce a decrease in exon 5N expression in these genes. In an embodiment, the ASOs useful for inducing an alteration in the splicing of exon 5N of the SCN8A gene include ASO-3 of nucleic acid sequence ctgcagaatcaaacca; ASO-8 of nucleic acid sequence gaaagtgcgtagagct; ASO-9 of nucleic acid sequence ctgaaagtgcgtagag; ASO-10 of sequence CCCTGAAAGTGCGTAG; and ASO-12 of nucleic acid sequence agtaccctgaaagtgc.

### Example 5: Identification of ASOs that Switch the Splicing of Exon 5 in the SCN8A Gene

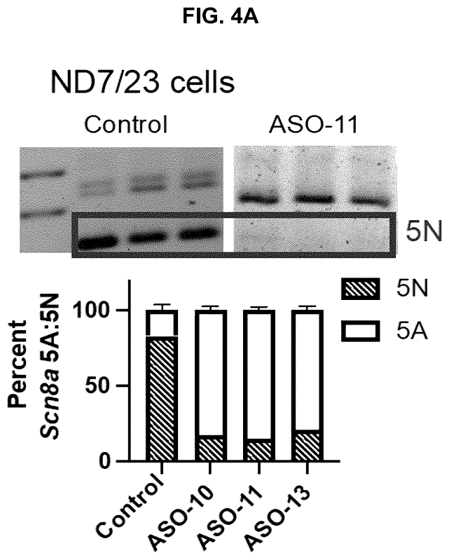

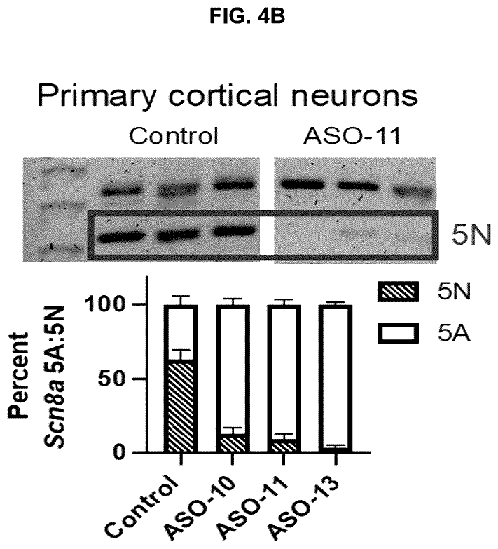

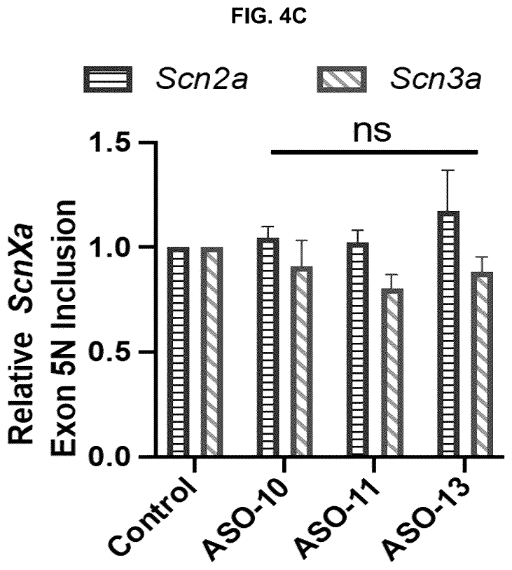

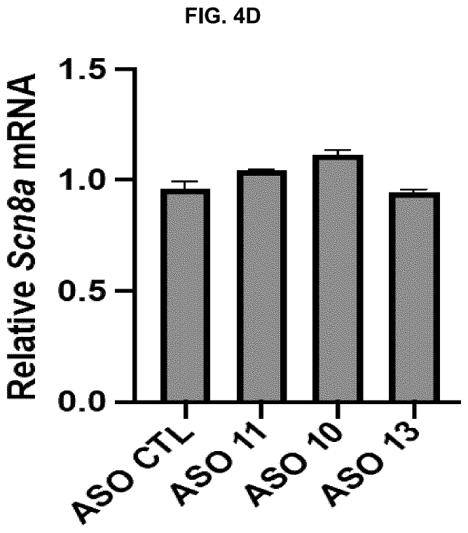

Further experiments were performed to identify ASOs that could induce a switch in splicing from 5N to 5A and from 5A to 5N in the mouse SCN8A gene. First, screening was performed to identify ASOs that induced a switch from exon 5N to 5A by tiling exon 5N and the intronic regions surrounding the exon. To measure the ratio of 5N:5A in cells (e.g., ND7/23 cells and primary cortical neurons), mRNA was extracted from the cell samples, and RT-PCR was performed. Each sample was then digested with two restriction enzymes, each of the enzymes cleaved only one exon (i.e., StyI for exon 5N and AvaII for exon 5A). Samples were electrophoresed on a gel, and the ratio of digested to undigested SCN8A was quantified from the image. The accuracy of this assay was validated using synthetic cDNAs mixed together at set ratios. All ASOs used in the experiments were 2′-MOE-modified 16-mers with phosphorothioate backbones. Based on this work, multiple ASOs that regulate exon 5 of SCN8A exon 5 splicing were identified. ASO-10, ASO-11, and ASO-13, which bind in the middle of exon 5N, induced a significant change in 5N inclusion, from over 85% at baseline in ND7/23 cells to less than 20% with the ASO (FIG. 4A). In primary cortical neurons isolated from the brains of P1 mice and cultured for 7 days, there was 60% 5N inclusion at baseline, and the same three ASOs reduced 5N levels to less than 10% (FIG. 4B). SCN8A has high homology with SCN1A, SCN2A and SCN3A, although the mouse SCN1A gene contains only exon 5A. The described ASOs had no significant effect on 5N inclusion in SCN2A and SCN3A (FIG. 4C), demonstrating the selectivity of these ASOs for SCN8A. In addition, it was confirmed that the ASOs had no effect on SCN8A mRNA expression (FIG. 4D). The results of these experiments validate three ASOs that induce a near complete switch from exon 5N to exon 5A in cultured cells and primary neurons without affecting the splicing of other Na⁺ channels or SCN8A mRNA expression.

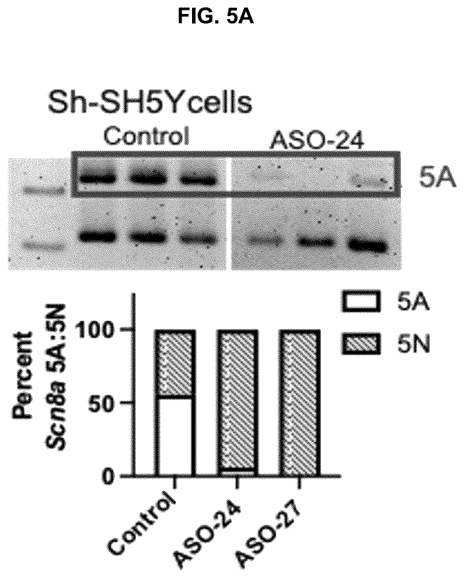

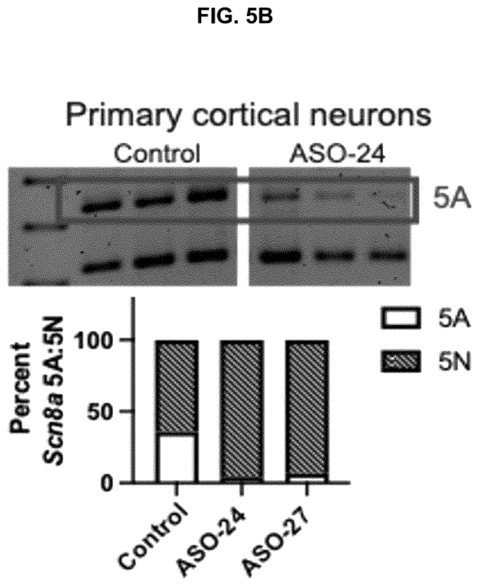

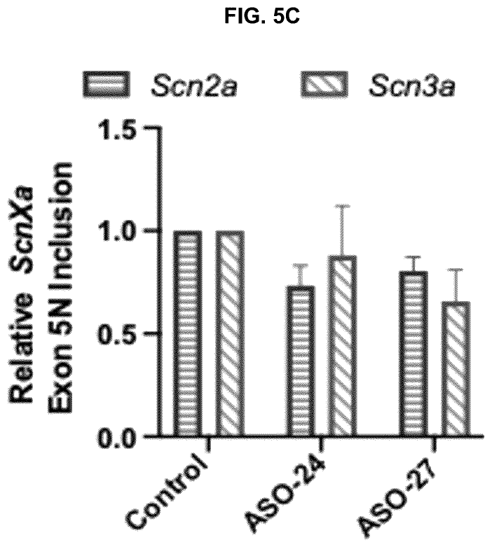

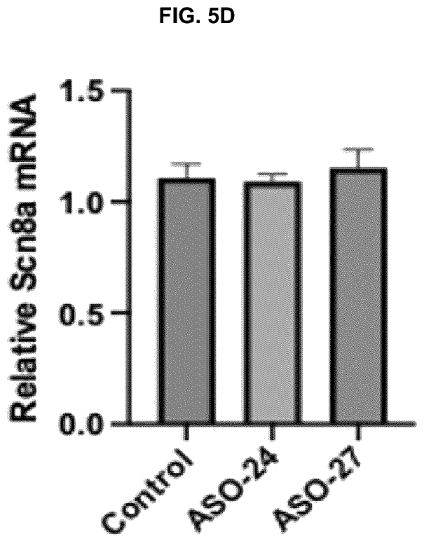

Another screen was performed to identify ASOs that could induce a switch in the opposite direction, that is, from 5A to 5N in SCN8A. Given the low level of 5A inclusion at baseline in ND7/23 cells, Sh-SH5Y cells were used. Sh-SH5Y cells are human neuroblastoma cells of the Sh-SH5Y human neuroblastoma cell line, which have just over 50% exon 5A inclusion in SCN8A at baseline, taking advantage of the fact that human and mouse exons 5A and 5N are 100% identical. ASOs were identified that regulated the alternative splicing of exon 5 of SCN8A. Two representative ASOs, namely, ASO-24 (nucleic acid sequence: atgttctcagcgctga) and ASO-27 (nucleic acid sequence: ggagaaccctgaatgt), which bind to the end of exon 5A, induced a significant change in exon 5A inclusion, in particular, from 50% to less than 8% (FIG. 5A). The nucleic acid sequences of ASO-24 and ASO-27 are presented in Table 3, below, with the backbone sequences of the ASOs.

TABLE 3

| | | Nucleic acid |
|---|---|---|
| | | sequence |
| ASO name | Nucleic acid sequence with backbone | (5′ to 3′) |
| scn8a_ASO-24 | /52MOErA/*/12MOErT/*/12MOErG/*/12MOErT/*/12MOE | atgttctcagcgctga |
| | rT/*/12MOErC/*/i2MOErT/*/i2MOErC/*/i2MOErA/*/i2M | |
| | OErG/*/12MOErC/*/i2MOErG/*/i2MOErC/*/12MOErT/*/ | |
| | i2MOErG/*/32MOErA/ | |
| scn8a_ASO-27 | /52MOErG/*/i2MOErG/*/12MOErA/*/12MOErG/*/12MOE | ggagaaccctgaatg |
| | rA/*/12MOErA/*/12MOErC/*/i2MOErC/*/i2MOErC/*/12 | t |
| | MOErT/*/12MOErG/*/12MOErA/*/i2MOErA/*/i2MOErT/ | |
| | */12MOErG/*/32MOErT/ | |

In primary cortical neurons isolated from the brains of P1 mice, 40% exon 5A inclusion at baseline was determined. The two ASOs reduced exon 5A levels to less than 10% in primary mouse cortical neurons (FIG. 5B). These ASOs had no significant effects on the inclusion of exon 5A in SCN2A and SCN3A (FIG. 5C) or on SCN8A mRNA expression (FIG. 5D). The results of the experiments validated the identification of representative ASOs that induce a near complete switch from exon 5A to exon 5N in cultured cells and primary neurons without affecting the splicing of other Na⁺ channels or SCN8A mRNA expression.

### Example 6: In Vivo Safety and Efficacy of Exon 5N-Targeting ASOs

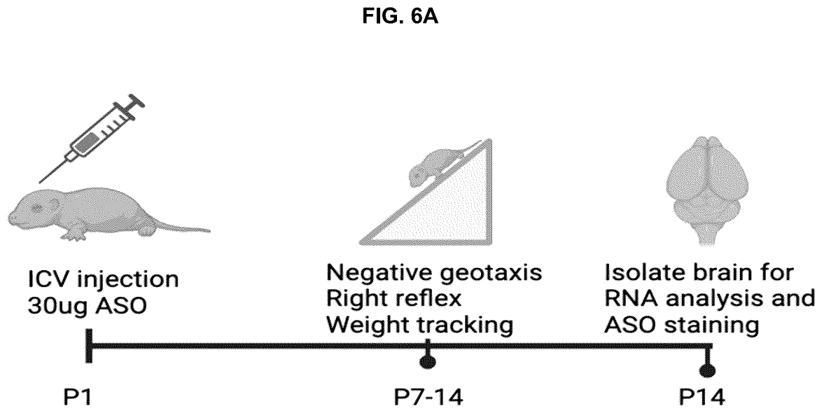

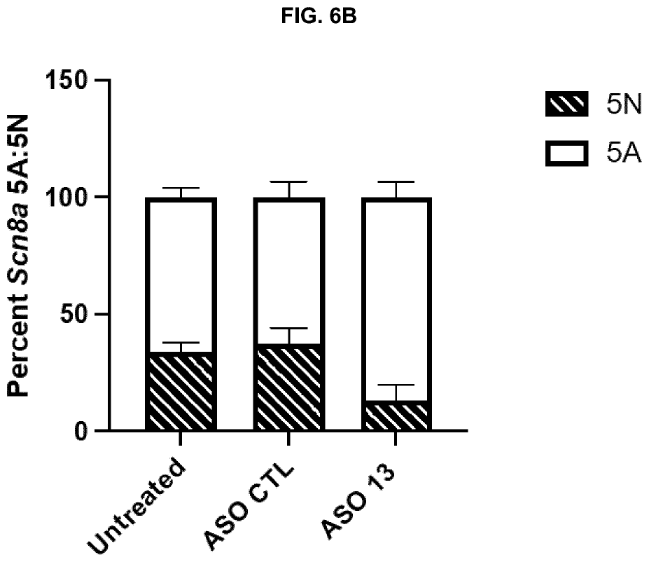

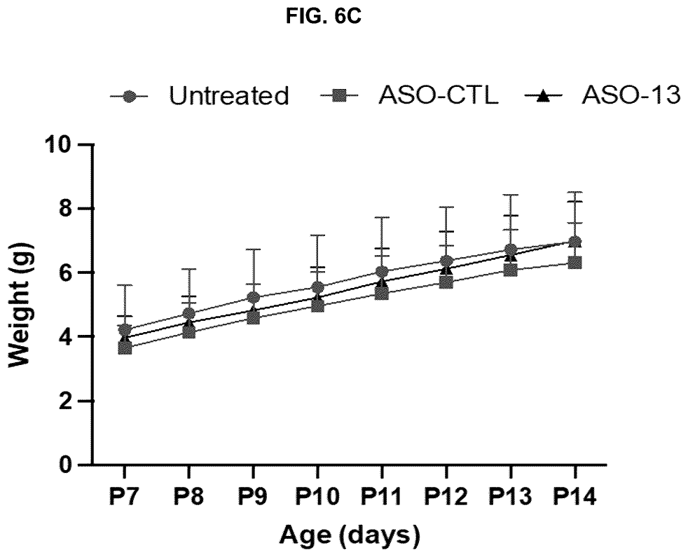

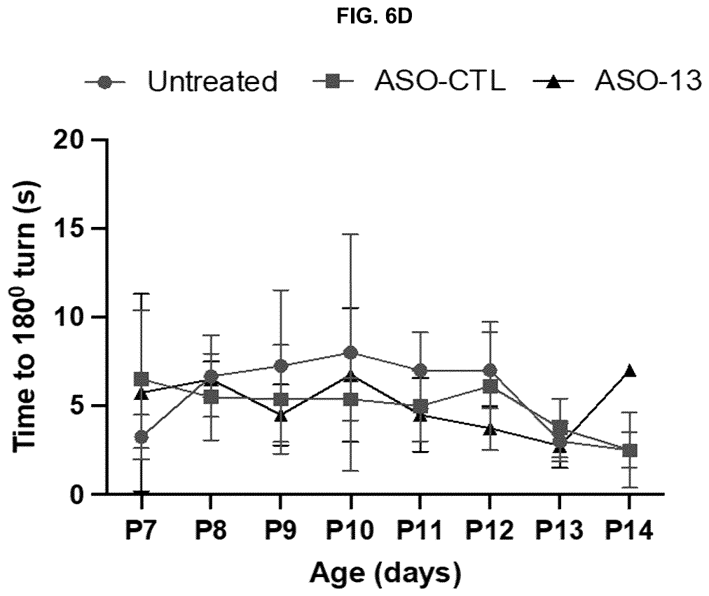

To determine the efficacy and safety of exon 5N-targeting ASOs in vivo, ASO-13 was administered via intracerebroventricular (ICV) injection to P1 C57BL/6 pups in an amount of 30 μg, 15 μg per ventricle. in 1 μL volumes. ASO-13 targets exon 5N of SCN8A with the goal of preventing the splicing machinery from binding and thus decreasing the expression of the 5N isoform. ASO-treated pups starting at P7 were weighed daily and assessed using developmental reflex assays. Fourteen days post injection, the treated and untreated mice were sacrificed, and brains were isolated from the animals. RNA was extracted from the brains before performing 5A:5N ratio assays. (FIG. 6A). Pups treated with ASO-13 had a decrease in 5N expression compared to untreated and control ASO (ASO CTL) treated pups (FIG. 6B). No difference was found in the weights of the pups treated with the two ASOs, thus demonstrating that the ASOs were not toxic (FIG. 6C). In addition, no difference was found between untreated and treated pups in the negative geotaxis assay, showing that pups treated with the ASOs had normal reflex development (FIG. 6D).

### Other Embodiments

From the foregoing description, it will be apparent that variations and modifications may be made to the aspects and embodiments as described herein to adopt them to various usages and conditions. Such embodiments are also within the scope of the claims that follow.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An antisense oligonucleotide (ASO) comprising 8-20 nucleobases, wherein at least 90% of the nucleobases or more than 8 consecutive nucleobases of the oligonucleotide are complementary to a nucleic acid sequence in an isoform of exon 5 of an SCN8A gene encoding a voltage-gated sodium ion channel polypeptide.

2. The antisense oligonucleotide of claim 1, wherein the isoform of exon 5 is the exon 5N isoform or the exon 5A isoform.

3. The antisense oligonucleotide of claim 1, wherein the antisense oligonucleotide comprises 10-16 consecutive nucleobases.

4. The antisense oligonucleotide of claim 1, wherein the voltage-gated sodium ion channel encoded by the SCN8A gene is Nav1.6.

5. The antisense oligonucleotide of claim 1, wherein the antisense oligonucleotide comprises or consists of a nucleic acid sequence having at least 80%, 90%, or 100% sequence identity to an ASO nucleotide sequence selected from the group listed in Table 1 or Table 2.

6. The antisense oligonucleotide of claim 1, wherein the antisense oligonucleotide comprises at least 10 consecutive nucleobases of the following nucleic acid sequences, or comprises or consists of the following nucleic acid sequences: cttgtaatggtatcac; aatggtaccactgggc; tgcagaatcaaacca; gcctaggtttacaaac; gcgtagagctgaaaca; gtgcgtagagctgaaa; aagtgcgtagagctga; gaaagtgcgtagagct; ctgaaagtgcgtagag; ccctgaaagtgcgtag; accctgaaagtgcgta; agtaccctgaaagtgc; ccctcagtaccctgaa; gggattaccgaaatag; ctgggattaccgaaa; acccaacacctgacac; ctaacccaacacctga; gccctaacccaacacc, atgttctcagcgctga or ggagaaccctgaatgt;
selected from ctgcagaatcaaacca; gcgtagagctgaaaca; gtgcgtagagctgaaa; aagtgcgtagagctga; gaaagtgcgtagagct; ctgaaagtgcgtagag; ccctgaaagtgcgtag; accctgaaagtgcgta; agtaccctgaaagtgc; or ccctcagtaccctgaa; or
selected from ctgcagaatcaaacca; gaaagtgcgtagagct; ctgaaagtgcgtagag; ccctgaaagtgcgtag; or agtaccctgaaagtgc.

7. An isolated or purified antisense oligonucleotide (ASO) for modifying pre-mRNA splicing of exon 5 in the SCN8A or the SCN5A sodium ion channel-encoding gene which specifically modulates splicing of the SCN8A exon 5N or 5A transcript or SCN5A exon 5N transcript, respectively.

8. A set or panel of antisense oligonucleotides (ASOs) comprising 2 or more of the antisense oligonucleotides of claim 1.

9. A pharmaceutical composition comprising an effective amount of the antisense oligonucleotide of claim 1.

10. A method of altering RNA splicing of exon 5, downregulating the activity of an exon 5 isoform of an SCN8A sodium ion channel gene in a cell, the method comprising:
contacting the cell with an effective amount of the antisense oligonucleotide of claim 1, thereby downregulating the exon 5 isoform activity.

11. A method of treating a neuronal, neurological, neurodevelopmental, or neurodegenerative disorder, disease, or pathology or symptoms thereof, associated with a variant or mutated gene encoding a voltage gated sodium ion channel protein in a subject, the method comprising:
administering to the subject an effective amount of the antisense oligonucleotide of claim 1 or a pharmaceutically acceptable composition thereof.

12. A method of inducing an alteration in RNA splicing of exon 5 isoforms of a gene encoding a voltage-gated sodium ion channel polypeptide in a cell of a subject; the method comprising:
administering to the subject an effective amount of the antisense oligonucleotide of claim 1 or a pharmaceutically acceptable composition thereof.

13. The method of claim 12, wherein the gene is an SCN8A polynucleotide or an SCN5A polynucleotide.

14. A method of promoting transcription of exon 5A or exon 5N of a gene encoding a voltage gated sodium ion channel protein in subject in need thereof, the method comprising:
administering a therapeutic agent to the subject in need thereof, wherein the therapeutic agent promotes exclusion of exon 5N or exon 5A transcription in the cell and promotes transcription of exon 5A or exon 5N and expression of the voltage gated sodium ion channel protein encoded by exon 5A or exon 5N; and
wherein the therapeutic agent is an antisense oligonucleotide of claim 1 or a pharmaceutically acceptable composition thereof.

15. A method for manipulating splicing of exon 5N or exon 5A of an SCN8A or SCN5A gene transcript, the method comprising:
providing an antisense oligonucleotide of claim 1, or a pharmaceutically acceptable composition thereof, and allowing the oligonucleotide to bind to a target exon 5N or exon 5A nucleic acid site.

16. The method of claim 15, wherein the antisense oligonucleotide induces an increase in exclusion of exon 5N or of exon 5A of the SCN8A gene in a cell.

17. The method of claim 15, wherein the antisense oligonucleotide induces a decrease in inclusion of exon 5N or of exon 5A of the SCN5A gene in a cell.

18. A cell comprising an antisense oligonucleotide of claim 1 and exon 5 of a gene encoding a voltage gated sodium ion channel protein.

19. The cell of claim 18, wherein the gene encoding a voltage gated sodium ion channel protein is SCN8A or SCN5A.

20. A kit or article of manufacture comprising the antisense oligonucleotide of claim 1, packaged in a suitable container, and directions for administering the antisense.
